(19) United States
(12) Patent Application Publication
(10) Pub. No.: US 2018/0296190 A1
(43) Pub. Date: Oct. 18, 2018

(54) ULTRASONIC DIAGNOSTIC DEVICE AND ULTRASONIC SIGNAL PROCESSING METHOD

(71) Applicant: Konica Minolta, Inc., Tokyo (JP)

(72) Inventor: Yasuaki SUSUMU, Osaka-shi, Osaka-fu (JP)

(21) Appl. No.: 15/765,876

(22) PCT Filed: Oct. 6, 2016
(86) PCT No.: PCT/JP2016/079831

§ 371 Date: Apr. 4, 2018

(30) Foreign Application Priority Data
Oct. 8, 2015 (JP) 2015-200488

(51) Int. Cl. A61B 8/08; A61B 8/00; G01S 7/52

(52) U.S. Cl. CPC A61B 8/485; A61B 8/085; A61B 8/463; A61B 8/469; G01S 7/52042; A61B 8/5269; A61B 8/4488; G01S 7/52022; A61B 8/5223

(57) ABSTRACT

An ultrasonic diagnostic device including a push pulse generator 104 that sets a specific site in a subject and causes a plurality of transducers 101a to transmit a push pulse pp; a detection pulse generator 105 that causes multiple transmissions of a detection pulse pwi that converges outside and passes through a region of interest roi in the subject; a displacement detector 109 that generates an acoustic line signal for each observation point Pij in the region of interest roi in order to detect displacement of tissue in the region of interest roi from an acoustic line signal frame data dsi sequence; and an elastic modulus calculator 110 that generates a wavefront frame data wfi sequence representing shear wave wavefront position, and calculates shear wave propagation speed and/or elastic modulus frame data emk in the region of interest roi based on the wavefront frame data wfi sequence.

### TECHNICAL FIELD

The present disclosure relates to ultrasonic diagnostic devices and ultrasonic signal processing methods, and in particular to tissue elastic modulus measurement using shear waves.

### BACKGROUND ART

An ultrasonic diagnostic device is a medical examination device that transmits ultrasound from transducers that constitute part of an ultrasound probe to the inside of a subject, receives ultrasonic reflected waves (echoes) caused by a difference in acoustic impedance of tissue in the subject, and generates and displays an ultrasound tomographic image showing structure of internal tissue of the subject based on electric signals obtained.

In recent years, tissue elastic modulus measurement applying this ultrasonic diagnostic technique (shear wave speed measurement (SWSM) hereinafter also referred to as “ultrasound elastic modulus measurement”) is being widely used for examination. This can non-invasively and easily measure hardness of tumor masses found in organs and body tissues, and is therefore useful in investigating tumor hardness in cancer screening tests and evaluating hepatic fibrosis in examination of liver disease.

In such ultrasound elastic modulus measurement, a region of interest (ROI) is determined in a subject, and a push pulse (converged ultrasonic wave or acoustic radiation force impulse (ARFI)) is transmitted to a specific site in the subject by converging ultrasound from a plurality of transducers, after which ultrasonic waves for detection (hereinafter also referred to as “detection pulses”) are transmitted and reflected waves are received multiple times. It is thereby possible to calculate propagation speed of a shear wave generated by acoustic radiation force of the push pulse by conducting propagation analysis of the shear wave, which represents elastic modulus of tissue, in order to display distribution of tissue elasticity as an elasticity image, for example (for examples, see Patent Literature 1 and 2).

### CITATION LIST

### Patent Literature

[Patent Literature 1] U.S. Pat. No. 7,252,004
[Patent Literature 2] U.S. Pat. No. 7,374,538

### SUMMARY OF INVENTION

### Technical Problem

In connection with examination by ultrasound elastic modulus measurement, there is a demand for an increase in time resolution of elasticity image acquisition and update speed of tissue elasticity images, and an increase in signal to noise (S/N) ratio of signals for elasticity image acquisition to improve image quality of the elasticity image, in order to facilitate confirmation of detailed changes in a lesion.

However, when using a plane wave in which an ultrasonic beam is transmitted in parallel from a plurality of transducers as a detection pulse as in the configuration described in Patent Literature 1, although it is possible to acquire a signal in a region of interest via one transmission and reception and thereby increase time resolution of signal acquisition, there is a technical problem that improvement of signal S/N cannot be achieved. On the other hand, according to the technique described in Patent Literature 2 of using converging ultrasound in which an ultrasonic beam is focused as a detection pulse, although signal S/N is increased, there is a technical problem that time resolution of signal acquisition cannot be improved because the number of times of transmission and reception to acquire a signal from a region of interest is large. Therefore an aim is to further improve time resolution of elasticity image acquisition and image quality of elasticity images through improvement of signal S/N.

The present disclosure is achieved in view of the technical problems described, and an aim of the present disclosure is to provide an ultrasonic diagnostic device and an ultrasonic signal processing method capable of improving signal acquisition time resolution and signal S/N for elasticity image generation in ultrasound elastic modulus measurement.

### Solution to Problem

An ultrasonic diagnostic device pertaining to one aspect of the present disclosure is an ultrasonic diagnostic device that causes a probe to transmit a push pulse converging on a specific site in a subject and detects propagation speed of a shear wave generated by acoustic radiation force of the push pulse, the probe being connectable to the ultrasonic diagnostic device and including transducers arranged along an array direction, the ultrasonic diagnostic device comprising: an operation input unit that receives operation input; a region of interest setter that sets a region of interest representing a range of analysis in the subject, based on the operation input; a push pulse generator that sets the specific site in the subject and causes the transducers to transmit the push pulse; a detection pulse generator that, following the push pulse, causes a portion of or all of the transducers to transmit detection pulses that each converge outside the region of interest and pass through the region of interest; a reception beamformer that generates an acoustic line signal frame data sequence by generating acoustic line signals with respect to observation points in the region of interest, based on reflected detection waves corresponding to the detection pulses that are reflected from tissue of the subject and received in a time sequence by the transducers; and an elastic modulus calculator that detects tissue displacement in the region of interest from the acoustic line signal frame data sequence, generates a wavefront frame data sequence representing shear wave wavefront position at time points on a time axis each corresponding to one of the detection pulses, and calculates shear wave propagation speed and/or elastic modulus frame data for the region of interest based on wavefront position changes and time intervals between the wavefront frame data frames.

### Advantageous Effects of Invention

According to the ultrasonic diagnostic device and the ultrasonic signal processing method pertaining to aspects of the present disclosure, signal acquisition time resolution and signal S/N for elasticity image generation can be improved in ultrasound elastic modulus measurement.

### BRIEF DESCRIPTION OF DRAWINGS

FIG. 1 is a schematic diagram illustrating an outline of a shear wave speed (SWS) sequence according to an ultrasound elastic modulus measurement method of ultrasonic diagnostic device 100 pertaining to Embodiment 1.

FIG. 2 is function block diagram of an ultrasonic diagnostic system 1000 including ultrasonic diagnostic device 100.

FIG. 3A and FIG. 3B are schematic diagrams illustrating schematic configurations of push pulses generated by push pulse generator 104.

FIG. 4 is a schematic diagram illustrating a schematic configuration of a detection pulse generated by the detection pulse generator 105.

FIG. 5A is a function block diagram illustrating configuration of transmission beamformer 106, and FIG. 5B is a function block diagram illustrating configuration of reception beamformer 108.

FIG. 6 is a schematic diagram illustrating an outline of an ultrasound propagation path calculation method of delay processing section 10831 of reception beamformer 108.

FIG. 7 is a function block diagram illustrating a configuration of displacement detector 109 and elastic modulus calculator 110.

FIG. 8 is a schematic diagram illustrating an outline of a process of a SWS sequence in ultrasonic diagnostic device 100.

FIG. 9 is a flowchart illustrating an operation of ultrasound elastic modulus calculation of ultrasonic diagnostic device 100.

FIG. 10 is a schematic diagram illustrating an outline of a process of a SWS sequence in ultrasonic diagnostic device 100.

FIG. 11A to FIG. 11E are schematic diagrams illustrating generation of a shear wave by a push pulse pp.

FIG. 12 is a flowchart illustrating an operation of shear wave propagation analysis of ultrasonic diagnostic device 100.

FIG. 13A to FIG. 13F are schematic diagrams illustrating an operation of shear wave propagation analysis.

FIG. 14 is a flowchart illustrating a beamforming operation of reception beamformer 108.

FIG. 15 is a flowchart illustrating an acoustic line signal generation operation for an observation point Pij by detection beamformer 108.

FIG. 16 is a schematic diagram for describing an acoustic line signal generation operation for an observation point Pij by reception beamformer 108.

FIG. 17A and FIG. 17B are simulation images illustrating acoustic line signals generated based on detection pulses; FIG. 17A illustrates a result pertaining to a comparative example using a plane wave pulse as a detection pulse, and FIG. 17B illustrates a result of using a detection pulse pertaining to ultrasonic diagnostic device 100.

FIG. 18 illustrates results of maximum sound pressure of acoustic line signals on a center axis A of the region of interest roi in FIG. 17A and FIG. 17B; the broken line being results of the comparative example and the unbroken line being results of ultrasonic diagnostic device 100.

FIG. 19 is a schematic diagram illustrating a schematic configuration of a detection pulse generated by detection pulse generator 105 in ultrasonic diagnostic device 100A pertaining to Embodiment 2.

FIG. 20 is a schematic diagram illustrating an outline of SWS sequence processing composed from SWS subsequences in ultrasonic diagnostic device 100A.

FIG. 21 is a schematic diagram illustrating an outline of a reception beamforming method of ultrasonic diagnostic device 100A.

FIG. 22 is a flowchart illustrating an ultrasound elastic modulus calculation operation of ultrasonic diagnostic device 100A.

FIG. 23 is a schematic diagram illustrating a schematic configuration of a detection pulse of an ultrasonic beam converging at transmission focus point F at a position shallower than region of interest roi, generated by detection pulse generator 105 in ultrasonic diagnostic device 100B.

FIG. 24A and FIG. 24B are schematic diagrams for describing an outline of a reception beamforming method of ultrasonic diagnostic device 100B and an acoustic line signal generation operation for observation point Pij in region of interest roi.

FIG. 25 is a flowchart illustrating a detection pulse generation operation of detection pulse generator 105 in ultrasonic diagnostic device 100B.

### DESCRIPTION OF EMBODIMENTS

### Embodiment 1

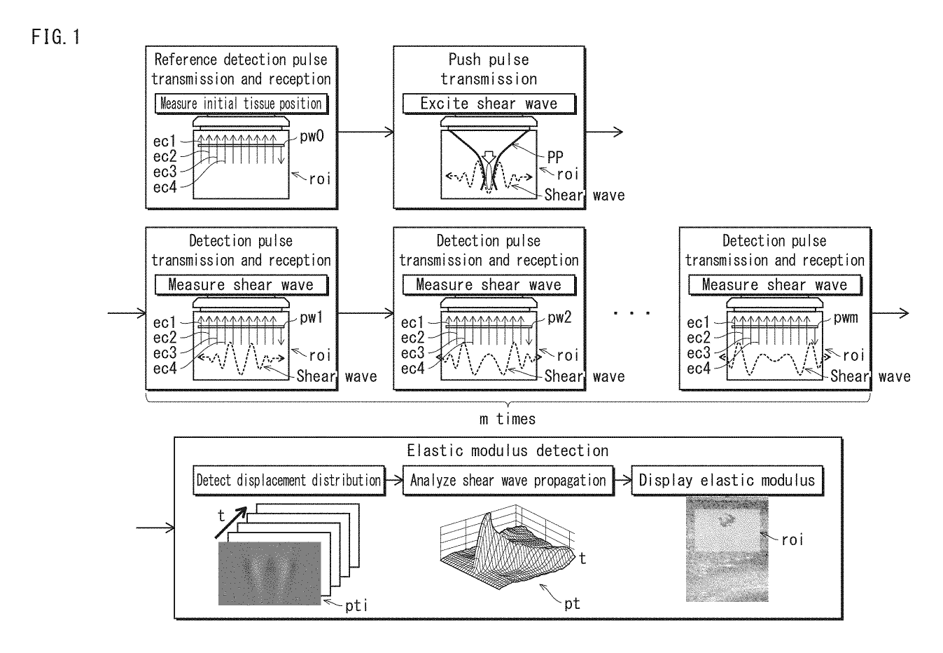

An ultrasonic diagnostic device 100 calculates shear wave propagation speed representing tissue elastic modulus, according to an ultrasound elastic modulus measurement method. FIG. 1 is a schematic diagram illustrating an outline of a shear wave speed (SWS) sequence according to an ultrasound elastic modulus measurement method in the ultrasonic diagnostic device 100. As illustrated in FIG. 1, processing of the ultrasonic diagnostic device 100 includes “reference detection pulse transmission and reception”, “push pulse transmission”, “detection pulse transmission and reception”, and “elastic modulus calculation”.

In “reference detection pulse transmission and reception”, transmission of detection pulse pw0 to a region of interest representing an analysis target range in a subject and reception of reflected waves ec1-ec4 is performed, generating an acoustic line signal as an initial position reference. In “push pulse transmission”, a push pulse pp obtained by converging ultrasound from a plurality of transducers to a specific site in the subject is transmitted in order to excite a shear wave. In “detection pulse transmission and reception”, the shear wave is measured by repeating transmission of a detection pulse pwi (i being a natural number from 1 to a detection pulse transmission number m) and reception of reflection waves ec1-ec4. In “elastic modulus calculation”, first, tissue displacement distribution pt associated with propagation of the shear wave generated by acoustic radiation pressure of the push pulse is measured in a time series. Next, shear wave propagation analysis is performed for calculating propagation speed of the shear wave, which is representative of tissue elastic modulus, from changes in the time series of displacement distribution pt, and finally tissue elasticity distribution is, for example, imaged for elastic modulus display as an elasticity image.

As illustrated above, the series of processes associated with one shear wave excitation based on push pulse pp transmission is referred to as a “SWS subsequence”, and a process in which a plurality of “SWS subsequences” are integrated is referred to as a “SWS sequence”.

<Ultrasonic Diagnostic System 1000>

1. Configuration Outline

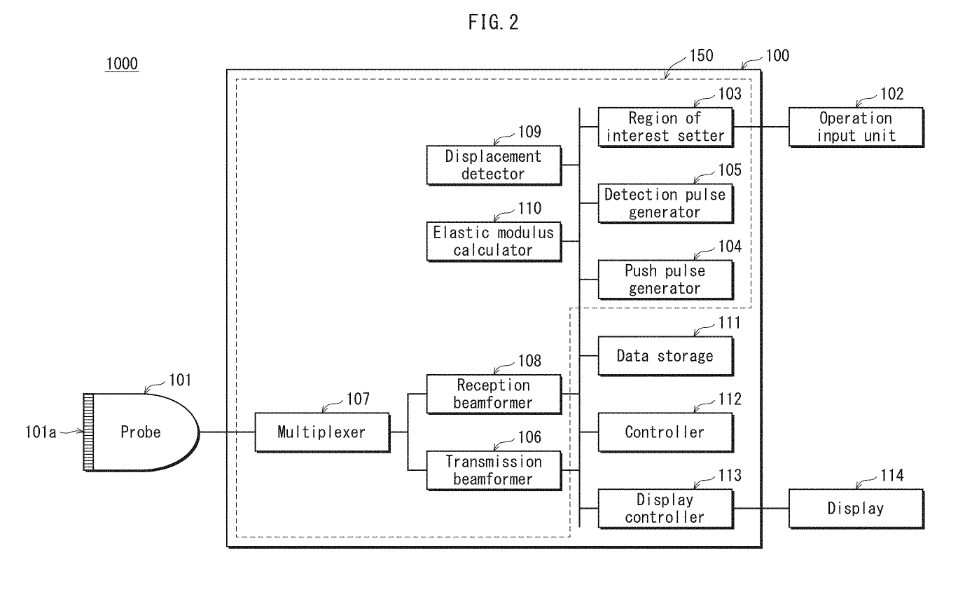

An ultrasonic diagnostic system 1000 including the ultrasonic diagnostic device 100 pertaining to Embodiment 1 is described with reference to the drawings. FIG. 2 is a function block diagram of the ultrasonic diagnostic system 1000 pertaining to Embodiment 1. As shown in FIG. 2, the ultrasonic diagnostic system 1000 includes: an ultrasound probe 101 (hereinafter also referred to as “probe 101”) that has a plurality of transducers 101a arranged on a front end surface thereof that transmit ultrasound towards a subject and receive reflected waves; the ultrasonic diagnostic device 100 that causes the probe 101 to transmit and receive ultrasound and generates an ultrasound image based on an output signal from the probe 101; an operation input unit 102 that receives operation input from a user; and a display 114 that displays the ultrasound image on a screen thereof. The probe 101, the operation input unit 102, and the display 114 are each connectable to the ultrasonic diagnostic device 100. FIG. 2 shows the probe 101, the operation input unit 102, and the display 114 connected to the ultrasonic diagnostic device 100. The probe 101, the operation input unit 102, and the display 114 may be functions included in the ultrasonic diagnostic device 100.

The following describes each element connected to the ultrasonic diagnostic device 100.

2. Probe 101

The probe 101 includes a plurality of transducers 101a arranged in, for example, a one-dimensional direction (hereinafter also referred to as “transducer array direction”). The probe 101 converts a pulsed electric signal (hereinafter also referred to as “transmit signal”) supplied from a transmission beamformer 106 (described later) into pulsed ultrasound. The probe 101, in a state in which a transducer-side outer surface of the probe 101 is in contact with a skin surface of a subject, transmits an ultrasonic beam composed of a plurality of ultrasonic waves emitted from a plurality of transducers towards a measurement target. Then the probe 101 receives a plurality of ultrasonic wave reflection waves (hereinafter also referred to as “reflected ultrasound”) from the subject, converts, via a plurality of transducers, the reflected ultrasound into electrical signals, and supplies the electrical signals to the reception beamformer 108.

3. Operation Input Unit 102

The operation input unit 102 receives various operation inputs such as various settings and operations with respect to the ultrasonic diagnostic device 100 from a user, and outputs to a controller 112 via a region of interest setter 103.

The operation input unit 102 may be, for example, a touch panel integrated with the display 114. In this case, various settings and operations of the ultrasonic diagnostic device 100 can be performed by touch operations and drag operations on operation keys displayed on the display 114, and the ultrasonic diagnostic device 100 is configured to be operable via the touch panel. Alternatively, the operation input unit 102 may, for example, be a keyboard that has various operation keys, various operation buttons, or an operation panel that has a lever or the like. Further, the operation unit 102 may be a trackball, mouse, touchpad, or the like for moving a cursor displayed on the display 114. Further, the operation unit 102 may use a plurality of the above, or be a combination of the above.

4. Display 114

The display 114 is a display device for image display, and displays an image output from a display controller 113 (described later) to a screen. A liquid crystal display, a cathode ray tube (CRT), an organic electroluminescence (EL) display, or the like can be used for the display 114.

<Configuration Outline of Ultrasonic Diagnostic Device 100>

The following describes the ultrasonic diagnostic device 100 pertaining to Embodiment 1.

The ultrasonic diagnostic device 100 includes: a multiplexer 107 that selects each transducer to be used when transmitting or receiving, among the transducers 101a of the probe 101, and secures input and output of selected transducers; the transmission beamformer 106 that controls timing of high voltage application to each of the transducers 101a of the probe 101 for ultrasound transmission; and the reception beamformer 108 that performs receive beamforming based on reflected waves received by the probe 101 in order to generate an acoustic line signal.

Further, the ultrasonic diagnostic device 100 includes: the region of interest setter 103 that sets a region of interest roi as a reference for a plurality of the transducers 101a, the region of interest roi representing an analysis target range in a subject, based on operation input from the operation input unit 102; a push pulse generator 104 that causes a plurality of the transducers 101a to transmit a push pulse, and a detection pulse generator 105 that causes multiple transmissions of a detection pulse after the push pulse.

Further, the ultrasonic diagnostic device 100 includes: a displacement detector 109 that detects tissue displacement in a region of interest roi from an acoustic line signal; and an elastic modulus calculator 110 that performs shear wave propagation analysis from detected tissue displacement and calculates shear wave propagation speed and/or elastic modulus in a region of interest roi.

Further, the ultrasonic diagnostic device 100 includes: a data storage 111 that stores an acoustic line signal outputted by the reception beamformer 108, displacement data outputted by the displacement detector 109, wavefront data and elastic modulus data outputted by the elastic modulus calculator 110, and the like; the display controller 113 that forms a display image and causes it to be displayed on the display 114; and the controller 112 that controls each element.

Of these elements, the multiplexer 107, the transmission beamformer 106, the reception beamformer 108, the region of interest setter 103, the push pulse generator 104, the detection pulse generator 105, the displacement detector 109, and the elastic modulus calculator 110 constitute ultrasonic signal processing circuitry 150.

Elements that constitute the ultrasonic signal processing circuitry 150, the controller 112, and the display controller 113 are each implemented by hardware circuitry such as field programmable gate arrays (FPGA), application specific integrated circuits (ASIC), or the like. Alternatively, such elements may be implemented by a programmable device such as a central processing unit (CPU), general-purpose computing on a graphics processing unit (GPGPU), a processor, or the like and software. These elements can each be a single circuit component or an assembly of circuit components. Further, a plurality of elements can be combined into a single circuit component or can be an aggregate of a plurality of circuit components.

The data storage 111 is a computer-readable storage medium, and may be a flexible disk, hard disk, magneto-optical (MO), digital versatile disc (DVD), digital versatile disc random access memory (DVD-RAM), semiconductor memory, or the like. Further, the data storage 111 may be a storage device that is externally connectable to the ultrasonic diagnostic device 100.

The ultrasonic diagnostic device 100 pertaining to Embodiment 1 is not limited to the ultrasonic diagnostic device configuration illustrated in FIG. 1. For example, the multiplexer 107 may be unnecessary, or the transmission beamformer 106 and/or the reception beamformer 108, or a portion thereof, may be housed in the probe 101.

<Configuration of Elements of Ultrasonic Diagnostic Device 100>

The following describes function blocks included in the ultrasonic diagnostic device 100.

1. Region of Interest Setter 103

Typically, when a B mode image, which is a cross-section image of a subject acquired in real time by the probe 101, is being displayed on the display 114, a user, using the B mode image displayed on the display 114 as an index, specifies an analysis target range in the subject and inputs to the operation input unit 102. The region of interest setter 103 sets information specified by a user via the operation input unit 102 as input, and outputs to the controller 112. At such time, the region of interest setter 103 may set a region of interest roi that represents an analysis target range in a subject with reference to position of a transducer array formed from the transducers 101a of the probe 101. For example, a region of interest roi may be information indicating a partial region in a virtual plane including the transducer array of the transducers 101a.

2. Push Pulse Generator 104

The push pulse generator 104 inputs information indicating a region of interest roi from the controller 112, sets a transmission focus point F at which an ultrasonic beam converges at a defined position in the region of interest roi in a subject, and causes a plurality of the transducers 101a to transmit a push pulse. Alternatively, a defined position outside a region of interest roi that is near the region of interest roi may be set as the transmission focus point F. In the case of a setting near a region of interest roi, the transmission focus point F is set at a distance from the region of interest roi that allows a shear wave to arrive at the region of interest roi.

More specifically, the push pulse generator 104 determines, as described below and based on information indicating a region of interest roi, a position of the transmission focus point F of a push pulse and a transducer array to transmit the push pulse (hereinafter also referred to as “push pulse transmission transducer array Px”).

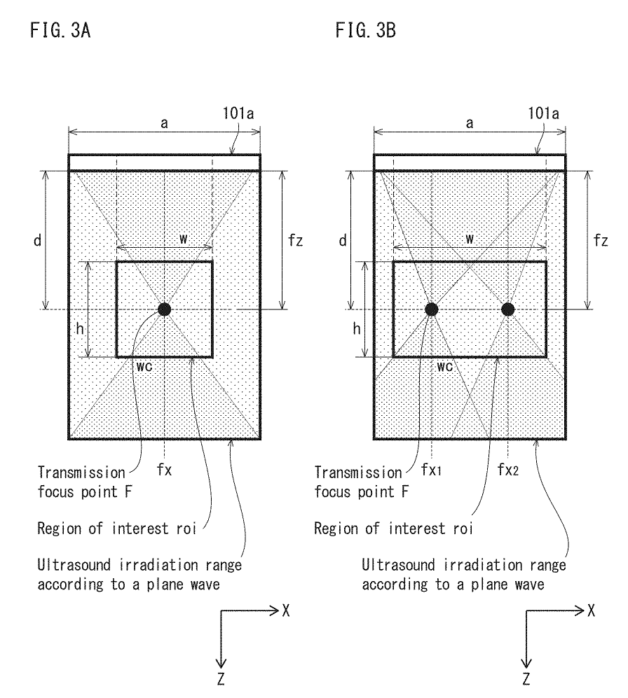

FIG. 3A and FIG. 3B are schematic diagrams illustrating schematic configurations of push pulses generated by the push pulse generator 104.

As illustrated in FIG. 3A, when a region of interest width w is small relative to a transducer array and array direction centers of both are coincident with each other, for position of transmission focus point F, an array direction transmission focus point position fx coincides with an array direction center position we of region of interest roi and a depth direction transmission focus point position fz coincides with a depth d to the center of the region of interest roi. Push pulse transmission transducer array length a is an array length of all of the transducers 101a.

As illustrated in FIG. 3B, when the region of interest width w is relatively large, a plurality of push pulses are generated. In this case, for positions of transmission focus points F, array direction transmission focus point positions fx1 and fx2 coincide with positions of internal divisions in the array direction of the width w of the region of interest roi, and a depth direction transmission focus point position fz coincides with a depth d to the center of the region of interest roi. Push pulse transmission transducer array length a is an array length of all of the transducers 101a.

Information indicating position of transmission focus point F and push pulse transmission transducer array Px is outputted to the transmission beamformer 106 as a transmission control signal together with pulse width of a push pulse.

3. Detection Pulse Generator 105

The detection pulse generator 105 inputs information indicating a region of interest roi from the controller 112, converges an ultrasonic beam to a transmission focus point F positioned outside the region of interest in a subject, sets the transmission focus point F so that an ultrasonic beam passes through the region of interest roi, and causes a plurality of the transducers 101a to transmit a detection pulse. More specifically, based on information indicating the region of interest roi, the detection pulse generator 105 determines, as indicated below, position of a transmission focus point F of a detection pulse and a transducer array to transmit the detection pulse (hereinafter also referred to as “detection pulse transmission transducer array Tx”).

Here, “converging” an ultrasonic beam according to a push pulse indicates that the ultrasonic beam is focused and is a focused beam, that is, an area irradiated by the ultrasonic beam decreases after transmission and achieves a minimum value at a specified depth, but does not indicate limitation to a case in which the ultrasonic beam is focused to a single point. The “transmission focus point F” indicates an ultrasonic beam center at a depth at which an ultrasonic beam converges.

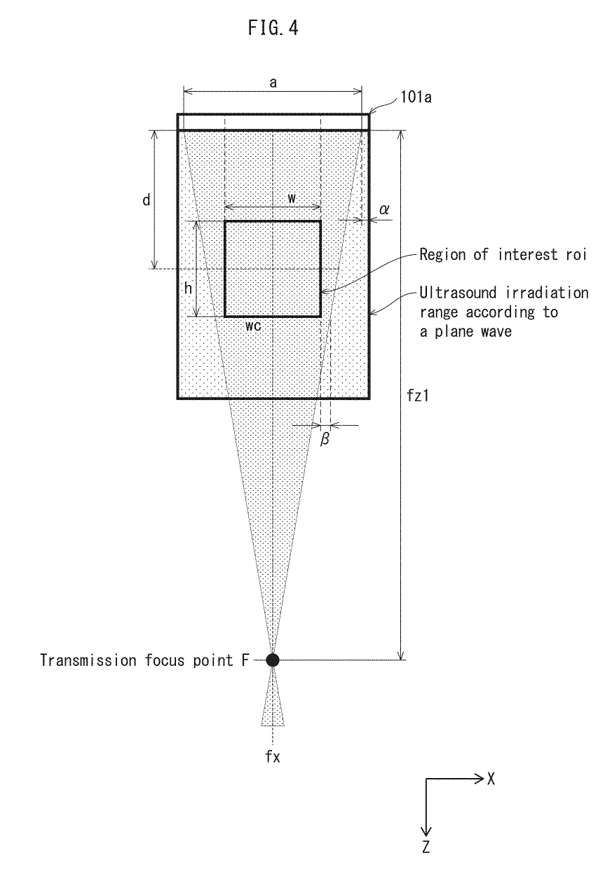

FIG. 4 is a schematic diagram illustrating a schematic configuration of a detection pulse generated by the detection pulse generator 105. As illustrated in FIG. 4, for a position of transmission focus point F, array direction transmission focus point position fx coincides with an array direction center position of region of interest roi. Further, depth direction transmission focus point position is at a depth fz1 such that an ultrasonic beam converges at transmission focus point F at a position deeper than the region of interest roi, outside the region of interest roi, and the ultrasonic beam passes through the entirety of the region of interest roi. Thus, an acoustic line signal can be generated for an observation point in the entirety of a region of interest by transmitting and receiving one detection pulse. For example, a region of interest roi may be configured to exist in a range sandwiched between two straight lines connecting the two ends of the detection pulse transmission transducer array Tx to a transmission focus point F, which is a beam center at a depth in a subject at which a detection pulse converges. Thus, the detection pulse can be transmitted such that an ultrasonic beam reliably passes through an entire region of interest.

Further, the detection pulse transmission transducer array Tx may be all of the transducers 101a. Further, in all SWS subsequences (1 to n) that constitute a SWS sequence, position of a transmission focus point F and the detection pulse transmission transducer array Tx are unchanging.

More specifically, when the number of the transducers 101a is nx, array direction pitch is k, and, as illustrated in FIG. 4, and a transmission transducer array length margin is α, array length a of the detection pulse transmission transducer array Tx satisfies:

[Math 1]

a=n×k−2α (Equation 1)

When region of interest roi array direction center position is wc, transmission focus point F array direction transmission focus point position fx satisfies:

[Math 2]

fx=w_{c} (Equation 2)

When depth from a subject surface to a region of interest roi center is d, length in the subject depth direction of the region of interest roi is h, array direction width of the region of interest roi is w, and an array direction distance representing a margin between a transmission beam and the region of interest roi is β, the margin being a distance between either of the two straight lines connecting an end of the detection pulse transmission transducer array Tx to a transmission focus point F in the subject (beam center at a depth at which the detection pulse converges), transmission focus point F depth direction transmission focus point position fz1 is preferably set to satisfy:

$$\begin{matrix}[ Math 3 ] & \\ fz 1 = \frac{a ( d + \frac{h}{2} )}{a - w - 2 β} & ( Equation 3 )\end{matrix}$$

Information indicating position of transmission focus point F and detection pulse transmission transducer array Tx is outputted to the transmission beamformer 106 as a transmission control signal together with pulse width of a detection pulse.

4. Transmission Beamformer 106

The transmission beamformer 106 is connected to the probe 101 via the multiplexer 107, and in order to transmit ultrasound from the probe 101, is a circuit that controls timing of application of a high voltage to all or a portion of the transducers 101a of the probe 101, or each of a plurality of transducers included in the push pulse transmission transducer array Px or the detection pulse transmission transducer array Tx.

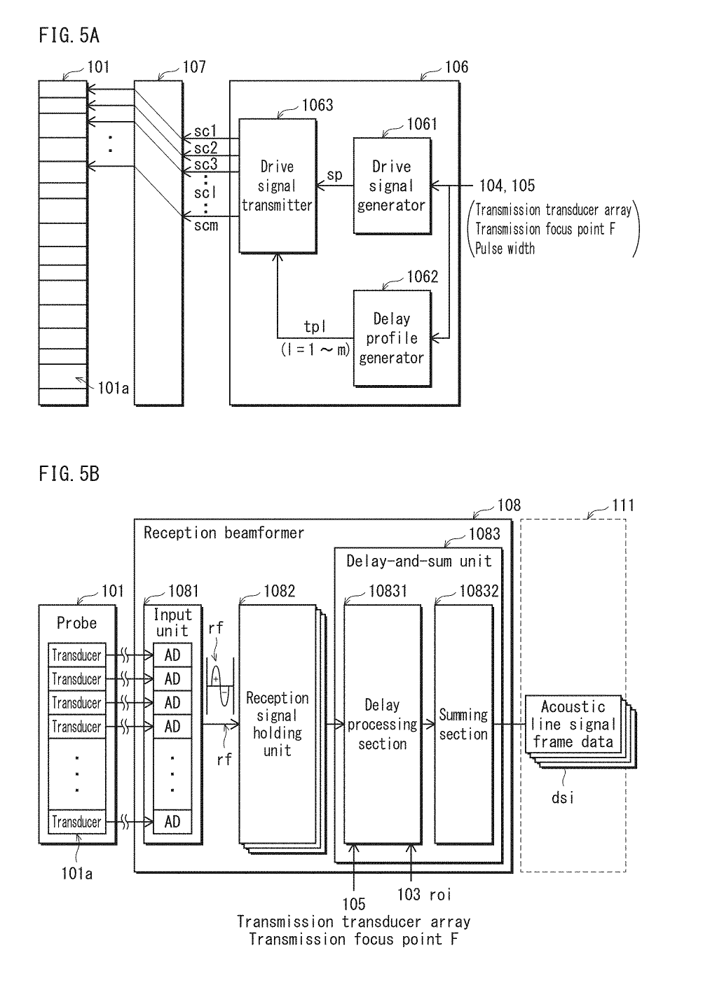

FIG. 5A and FIG. 5B are function block diagrams illustrating configuration of the transmission beamformer 106. As illustrated in FIG. 5A, the transmission beamformer 106 includes a drive signal generator 1061, a delay profile generator 1062, and a drive signal transmitter 1063.

(1) Drive Signal Generator 1061

The drive signal generator 1061 is a circuit that generates, among transmission control signals from the push pulse generator 104 and the detection pulse generator 105, a pulse signal sp for causing all or a portion of the transducers 101a of the probe 101 to transmit an ultrasonic beam, based on information indicating pulse width and the push pulse transmission transducer array Px or the detection pulse transmission transducer array Tx.

(2) Delay Profile Generator 1062

The delay profile generator is a circuit that, among transmission control signals obtainable from the push pulse generator 104 and the detection pulse generator 105, sets and outputs for each transducer a delay time tpl (l being a natural number from 1 to the number of detection pulse transmission transducers) that determines a transmission timing of an ultrasonic beam based on information indicating a transmission focus point F and the push pulse transmission transducer array Px or the detection pulse transmission transducer array Tx. Thus, ultrasonic beam focusing is performed by causing transmission of an ultrasonic beam to be delayed for each transducer by a delay time assigned thereto.

(3) Drive Signal Transmitter 1063

The drive signal transmitter 1063 is a circuit that performs transmission processing of supplying a transmission signal scl for causing transmission of an ultrasonic beam to each transducer included in the push pulse transmission transducer array Px or the detection pulse transmission transducer array Tx among the transducers 101a of the probe 101, based on a pulse signal sp from the drive signal generator 1061 and a delay time tpl from the delay profile generator 1062. The push pulse transmission transducer array Px or the detection pulse transmission transducer array Tx is selected by the multiplexer 107.

Based on a transmission control signal from the push pulse generator 104, the transmission beamformer 106 repeats push pulse transmission while gradually moving transmission focus point F in the array direction for each push pulse transmission, causing a shear wave to propagate for all regions in a region of interest roi. At this time, for example, the transmission beamformer 106 may be configured to move the transmission focus point F in the array direction by repeating push pulse transmission while gradually moving the push pulse transmission transducer array Px in the array direction for each push pulse transmission.

The transmission beamformer 106, after push pulse transmission, repeatedly transmits a detection pulse based on a transmission control signal from the detection pulse generator 105. After one push pulse transmission, one series of multiple detection pulse transmissions from the same detection pulse transmission transducer array Tx is referred to as a “transmission event”.

According to Embodiment 1, in all SWS subsequences included in a SWS sequence, the detection pulse transmission transducer array Tx is configured to be all of a plurality of the transducers 101a. However, as indicated by Embodiment 2, the transmission beamformer 106 may be configured to repeat detection pulse transmission while gradually moving the detection pulse transmission transducer array Tx of a detection pulse in the array direction for each SWS subsequence in order to perform detection pulse transmission from all the transducers 101a of the probe 101.

5. Reception Beamformer 108 Configuration

The reception beamformer 108 is a circuit that, based on reflected detection waves from tissue of a subject received in time series by a plurality of the transducers 101a corresponding to each of a plurality of detection pulses, generates an acoustic line signal for each of a plurality of observation points Pij in a region of interest roi to generate a sequence of acoustic line signal frame data dsi. That is, the reception beamformer 108, after detection pulse transmission, generates an acoustic line signal from electric signals obtained by a plurality of the transducers 101a, based on reflected waves received by the probe 101. Note that an “acoustic line signal” is a reception signal for an observation point after delay-and-sum processing.

FIG. 5B is a function block diagram illustrating configuration of the reception beamformer 108. The reception beamformer 108 includes an input unit 1081, a reception signal holding unit 1082, and a delay-and-sum unit 1083.

5.1 Input Unit 1081

The input unit 1081 is connected to the probe 101 via the multiplexer 107, and is a circuit that generates a reception signal (RF signal) based on reflected waves at the probe 101. Here, a reception signal rf (RF signal) is a digital signal obtained by A/D converting an electrical signal converted from reflected ultrasound received by each transducer based on transmission of a transmission signal scl, and is composed of a series of signals (reception signal sequence) that are continuous in transmission direction (subject depth direction) of ultrasound received by each transducer.

The input unit 1081 generates a reception signal rf sequence for each reception transducer for each transmission event, based on reflected ultrasound obtained by each reception transducer selected for SWS subsequence synchronization. A reception transducer array is composed from part or all of the transducers 101a of the probe 101, and is selected by the multiplexer 107 based on an instruction from the controller 116 for each SWS subsequence. According to the present example, all of a plurality of the transducers 101a are selected as a reception transducer array in all SWS subsequences included in a SWS sequence. This makes it possible to generate a reception transducer array of all transducers to receive reflected detection waves from a given observation point using all transducers, such that when delay-and-sum is performed, signal S/N of an acoustic line signal can be improved.

A generated reception signal rf is outputted to the reception signal holding unit 1082.

5.2 Reception Signal Holding Unit 1082

The reception signal holding unit 1082 is a computer-readable storage medium such as a semiconductor memory, for example. The reception signal holding unit 1082 inputs a reception signal rf for each reception transducer from the input unit 1081, synchronized to a transmission event, holding same until one frame of acoustic line signal frame data is generated for the transmission event.

The reception signal holding unit 1082 can make use of, for example, a hard disk, MO, DVD, DVD-RAM, or the like. Further, the reception signal holding unit 1082 may be a storage device that is externally connectable to the ultrasonic diagnostic device 100. Further, the reception signal holding unit 1082 may be a portion of the data storage 111.

5.3 Delay-and-Sum Unit 1083

The delay-and-sum unit 1083 is a circuit that, synchronized to a transmission event, after performing delay processing on reception signals rf from an observation point Pij in a region of interest roi and received by reception transducers Rpl included in the detection pulse reception transducer array Rx, performs summing for all the reception transducers Rpl to generate an acoustic line signal ds. The detection pulse reception transducer array Rx is composed from reception transducers Rpl that are part or all of the transducers 101a of the probe 101, and is selected by the delay-and-sum unit 1083 and the multiplexer 107 based on an instruction from the controller 112 for each SWS subsequence. According to the present example, a transducer array including at least all of the transducers of the detection pulse transmission transducer array Tx for one transmission event corresponding to a SWS subsequence is selected as the detection pulse reception transducer array Rx.

The delay-and-sum unit 1083 includes a delay processing section 10831 and a summing section 10832 for processing reception signals rf.

(1) Delay Processing Section 10831

The delay processing section 10831 is a circuit that identifies, from reception signals (reception signal sequences) corresponding to reception transducers Rpl in the detection pulse reception transducer array Rx, a reception signal as corresponding to a reception transducer Rpl, based on reflected ultrasound from an observation point Pij, by compensating according to an arrival time difference (delay) of the reflected ultrasound to the reception transducer Rpl, which is obtained by dividing a difference in distance between the observation point Pij and the reception transducer Rpl by a speed of sound value.

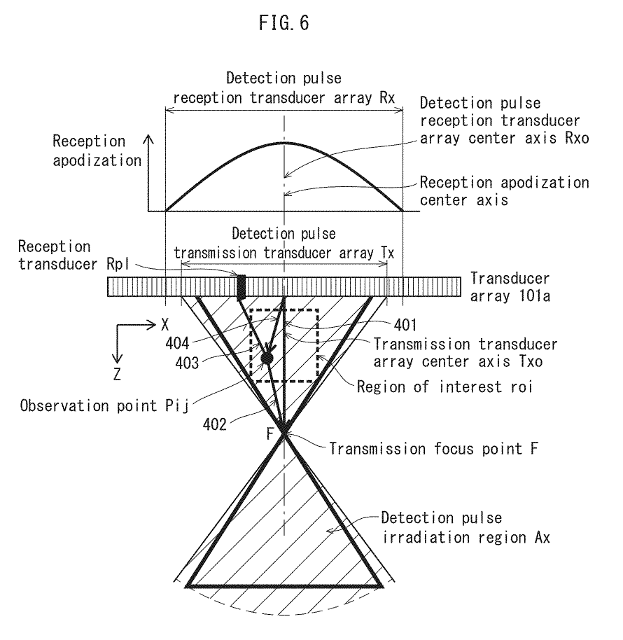

FIG. 6 is a schematic diagram illustrating an outline of an ultrasound propagation path calculation method in the delay processing section 10831 of the reception beamformer 108. FIG. 6 illustrates a propagation path of ultrasound radiated from the detection pulse transmission transducer array Tx and reflected at an observation point Pij at an arbitrary position in a region of interest roi to arrive at a reception transducer Rpl.

a) Transmission Time Calculation

First, the delay processing section 10831, in response to a transmission event, calculates a transmission path for transmitted ultrasound to arrive at an observation point Pij in a region of interest roi in a subject, based on information indicating transmission focus point F position and transducers included in the detection pulse transmission transducer array Tx acquired from the detection pulse generator 105, and information indicating position of the region of interest roi acquired from the region of interest setter 103, then divides by the speed of sound to calculate transmission time.

Here, a transmission path is assumed such that, after a detection pulse radiated from the detection pulse transmission transducer array Tx passes along a path 401 and a wave front thereof converges at transmission focus point F, the detection pulse reaches the observation point Pij in the region of interest roi at a position shallower than the transmission focus point F along a path 402. That is, a calculation is performed assuming that, when the observation point Pij is shallower than the transmission focus point F, a time at which a transmission wave radiated from the detection pulse transmission transducer array Tx arrives at the transmission focus point F along the path 401 and a time at which the transmission wave arrives at the transmission focus point F along the path 402 from the observation point Pij after arriving at the observation point Pij along a path 404 are the same. Accordingly, a value obtained by subtracting a travel time of a transmission wave along the path 402 from a travel time along the path 401 is the transmission time. As a specific calculation method, for example, a path length difference obtained by subtracting length of the path 402 from length of the path 401 can be divided by propagation speed of ultrasound in a subject in order to obtain the transmission time.

A transmission focus point F is defined as a design value by the detection pulse generator 105, and therefore length of the path 402 from the transmission focus point F to any observation point Pij can be geometrically calculated.

b) Reception Time Calculation

Next, the delay processing section 10831, in response to a transmission event, calculates a reception path for transmitted ultrasonic reflected at an observation point Pij in a region of interest roi to arrive at a reception transducer Rpl of the detection pulse reception transducer array Rx, based on information indicating position of the detection pulse reception transducer array Rx from the data storage 111, then divides by the speed of sound to calculate reception time.

More specifically, for calculation purposes a reception path is assumed such that, if there is a change in acoustic impedance at an observation point Pij, a reflected wave is generated, and the reflected wave travels along a path 403 to return to a reception transducer Rpl of the probe 101.

Position information of a reception transducer Rpl in the detection pulse reception transducer array Rx is obtained from the controller 112, and therefore length of the path 403 from any observation point Pij to the reception transducer Rpl can be geometrically calculated.

c) Delay Calculation

Next, the delay processing section 10831 calculates total propagation time to a reception transducer Rpl in the detection pulse reception transducer array Rx from transmission time and reception time, and calculates delay to be applied to a reception signal sequence corresponding to the reception transducer Rpl. That is, total propagation time until a transmitted ultrasonic wave arrives at a reception transducer Rpl via an observation point Pij is calculated, and delay to be applied to a reception signal sequence corresponding to the reception transducer Rpl is calculated according to differences in total propagation time among reception transducers Rpl.

d) Delay Processing

Next, the delay processing section 10831, from a reception signal sequence corresponding to a reception transducer Rpl in the detection pulse reception transducer array Rx, identifies a reception signal corresponding to a delay of a reception transducer Rpl (reception signal corresponding to a time from which delay is subtracted) as a reception signal corresponding to the reception transducer Rpl, based on reflected ultrasound from an observation point Pij.

The delay processing section 10831, in response to a transmission event, inputs reception signals rf from the reception signal holding unit 1082, and performs the processing described above for all observation points Pij in a region of interest roi.

(2) Summing Section 10832

The summing section 10832 is a circuit that inputs reception signals identified as corresponding to reception transducers Rpl and outputted from the delay processing section 10831, sums the reception signals, and generates an acoustic line signal subjected to delay-and-sum with respect to an observation point Pij.

Alternatively, as illustrated in FIG. 6, reception signals identified as corresponding to each reception transducer Rpl may be multiplied by a weighting sequence (reception apodization) corresponding to reception transducers and summed, in order to generate an acoustic line signal with respect to an observation point Pij. A weighting sequence is a weighting coefficient sequence applied to reception signals corresponding to reception transducers Rpl in the detection pulse reception transducer array Rx. A weighting sequence is set for reception transducers Rpl such that transducers positioned centrally in the array direction of the detection pulse reception transducer array Rx have a maximum weight and a central axis of distribution coincides with a detection pulse reception transducer array axis Rxo, and distribution has a symmetrical shape with respect to the central axis. As a shape of a weighting sequence, a Hamming window, a Hann window, a rectangular window, or the like can be used, and the shape of distribution is not particularly limited.

The summing section 10832, in response to a transmission event, generates an acoustic line signal for every observation point Pij present in a region of interest roi.

The delay processing section 10831 adjusts phase of reception signals detected by reception transducers Rpl in the detection pulse reception transducer array Rx and the summing section 10832 performs summing processing, and therefore reception signals received by reception transducers Rpl, based on reflected waves from an observation point Pij, can be superimposed to increase signal S/N ratio, and a reception signal from the observation point Pij can be extracted.

In response to one transmission event, acoustic line signals generated for all observation points Pij in a region of interest roi are acoustic line signal frame data dsi. Then, synchronized to transmission events, transmission and reception of detection pulses is repeated to generate acoustic line signal frame data for all transmission events.

Acoustic line signal frame data dsi (i being a natural number from 1 to a transmission event count number m) generated synchronized to a transmission event is stored in the data storage 111.

6. Displacement Detector 109

The displacement detector 109 is a circuit that detects displacement of tissue in a region of interest roi from a sequence of acoustic line signal frame data dsi.

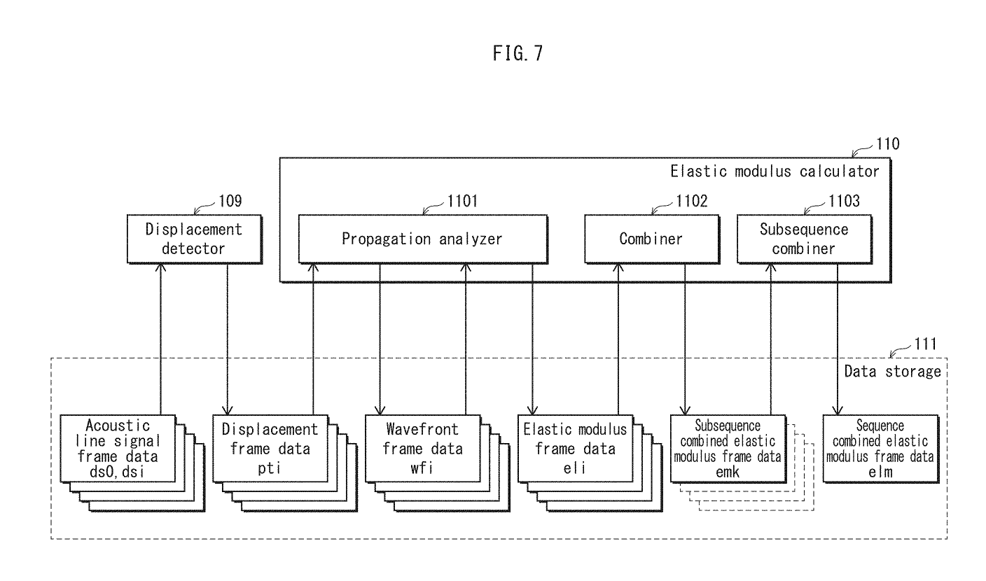

FIG. 7 is a function block diagram illustrating a configuration of the displacement detector 109 and the elastic modulus calculator 110. The displacement detector 109 acquires one frame of acoustic line signal frame data dsi as a target for displacement detection in a sequence of acoustic line signal frame data dsi and one frame of acoustic line signal frame data ds0 as a reference (hereinafter also referred to as “reference acoustic line signal frame data ds0”) from the data storage 111 via the controller 112. The reference acoustic line signal frame data ds0 is a reference signal for extracting displacement due to a shear wave in acoustic line signal frame data dsi corresponding to each transmission event, and more specifically is acoustic line signal frame data acquired from a region of interest roi prior to push pulse transmission. From the difference between acoustic line signal frame data dsi and reference acoustic line signal frame data ds0, the displacement detector 109 detects displacement (image information movement) of an observation point Pij in a region of interest roi of acoustic line signal frame data dsi, and associates displacement with observation point Pij coordinates to generate displacement frame data pti. The displacement detector 109 outputs generated displacement frame data pti to the data storage 111 via the controller 112.

7. Elastic Modulus Calculator 110

The elastic modulus calculator 110 includes a propagation analyzer 1101, a combiner 1102, and a subsequence combiner 1103.

7.1. Propagation Analyzer 1101

The propagation analyzer 1101 is a circuit that, for each SWS subsequence, from a displacement frame data pti sequence, generates wavefront frame data wfi representing shear wave wavefront position at a plurality of time points on a time axis corresponding to a plurality of detection pulses, and calculates shear wave propagation speed or elastic modulus frame data in a region of interest roi, based on amounts of change in wavefront position among a plurality of wavefront frame data wfi and time intervals between frames.

More specifically, the propagation analyzer 1101 acquires displacement frame data pti from the data storage 111 via the controller 112. The propagation analyzer 1101 detects position, travel direction, and speed of a shear wave from displacement data pti at each time displacement data pti is acquired, and generates a wavefront frame data wfi sequence. The propagation analyzer 1101 calculates elastic modulus data of tissue of a subject corresponding to an observation point Pij in a region of interest roi of displacement frame data pti from position, travel direction, and speed of a shear wave indicated by a wavefront frame data wfi sequence, and generates an elastic modulus frame data eli sequence. The propagation analyzer 1101 outputs generated wavefront frame data wfi and elastic modulus frame data eli to the data storage 111 via the controller 112.

7.2. Combiner 1102

The combiner 1102 combines shear wave propagation speed corresponding to a plurality of transmission events included in a SWS subsequence, or an elastic modulus frame data eli sequence, to calculate shear wave propagation speed of one frame corresponding to the SWS subsequence, or SWS subsequence combined elastic modulus frame data emk.

7.3. Subsequence Combiner 1103

The subsequence combiner 1103 combines shear wave propagation speed pertaining to a plurality of frames SWS subsequences included in a SWS sequence, or SWS subsequence combined elastic modulus frame data emk, to calculate shear wave propagation speed of one frame corresponding to a SWS sequence, or SWS sequence combined elastic modulus frame data elm.

8. Other Configuration

The data storage 111 is a storage medium that sequentially records generated reception signal series rf, acoustic line signal frame data dsi sequence, displacement frame data pti sequence, wavefront frame data wfi sequence, elastic modulus frame data eli sequence, subsequence combined elastic modulus frame data emk, and sequence combined elastic modulus frame data elm.

The controller 112 controls each block in the ultrasonic diagnostic device 100, based on instruction from the operation input unit 102. As the controller 112, a processor such as a CPU can be used.

Further, although not illustrated, the ultrasonic diagnostic device 100 has a B mode image generator that generates ultrasound images (B mode images) in a time series based on components reflected from tissue of a subject among acoustic line signals outputted based on transmission and reception of ultrasound by the transmission beamformer 106 and the reception beamformer 108, without transmission of a push pulse. The B mode image generator inputs acoustic line signal frame data from the data storage 111, performs processing such as envelope detection and logarithmic compression on the acoustic line signal to convert it to a luminance signal corresponding to intensity, then subjects the luminance signal to coordinate transformation to an orthogonal coordinate system to generate B mode image frame data. Note that ultrasound transmission and reception by the transmission beamformer 106 and the reception beamformer 108 for acquiring an acoustic line signal for B mode image generation can use a publicly-known method. Generated B mode image frame data is outputted to and stored by the data storage 111. The display controller 113 configures a B mode image as a display image and causes the display 114 to display the display image.

Further, the propagation analyzer 1101 may be configured to generate and display an elasticity image mapped to color information based on elastic modulus indicated by elastic modulus frame data eli. For example, an elasticity image may be generated in different colors in which coordinates at which elastic modulus is equal to or greater than a certain value are red, coordinates at which elastic modulus is less than the certain value are green, and coordinates at which elastic modulus could not be acquired are black. The propagation analyzer 1101 outputs generated elastic modulus frame data eli and an elasticity image to the data storage 111, and the controller 112 outputs an elasticity image to the display controller 113. Further, the display controller 113 may be configured to perform a geometric transformation on an elasticity image to transform it to image data for display, and output the post-geometric transformation elasticity image to the display 114.

<Operations>

The following describes SWS sequence operations of the ultrasonic diagnostic device 100 configured as described above.

1. Operations Outline

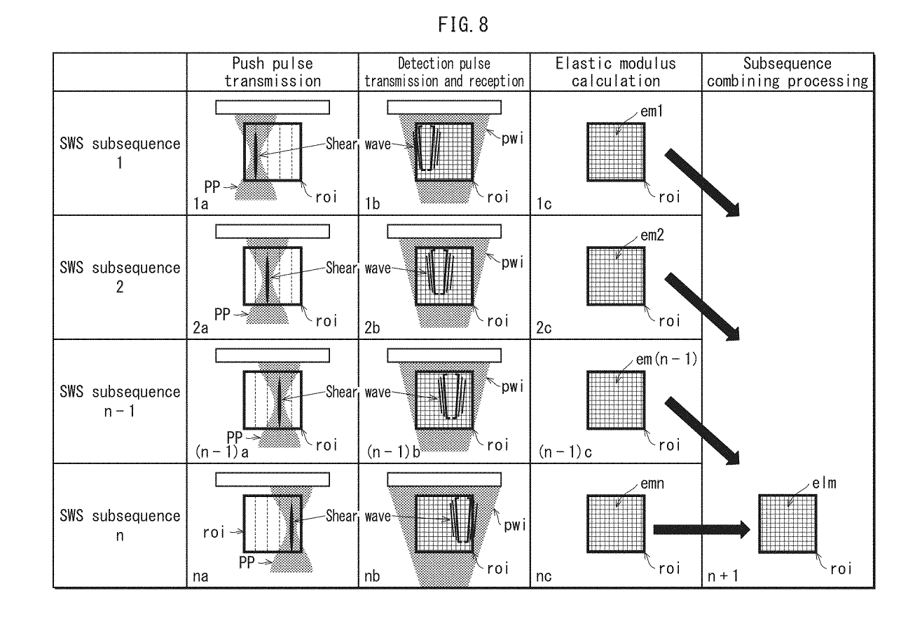

FIG. 8 is a schematic diagram showing an outline of an SWS sequence process in the ultrasonic diagnostic device 100. Tissue elastic modulus measurement by the ultrasonic diagnostic device 100 is performed from a SWS sequence including a plurality of SWS subsequences accompanying a single shear wave excitation based on push pulse pp transmission. As illustrated in FIG. 8, according to Embodiment 1, a SWS sequence is composed of n SWS subsequences.

SWS subsequences (1 to n) are composed from processes including: push pulse transmission of gradually moving a specific site at which a push pulse pp converges in the array direction for each subsequence to transmit a push pulse pp to the specific site in a subject to excite a shear wave; multiple (m) repetitions of detection pulse transmission and reception of detection pulse pwi transmission and reception with respect to a region of interest roi; and elastic modulus calculation of performing shear wave propagation analysis and calculating shear wave propagation speed and elastic modulus emk (k=1 to n).

In a SWS sequence, after a plurality of SWS subsequences (1 to n) are obtained, elastic modulus emk calculated for each SWS subsequence is combined by subsequence combining processing to calculate SWS sequence combined elastic modulus elm.

2. SWS Sequence Operations

The following describes an operation of ultrasound elastic modulus measurement processing after a B mode image is displayed on the display 114, in which tissue is drawn based on reflection components from tissue of a subject based on a publicly-known method.

B mode image frame data is generated without transmission of a push pulse, and acoustic line signal frame data is generated in a time series based on reflected components from tissue of a subject, based on transmission and reception of ultrasound by the transmission beamformer 106 and the reception beamformer 108, the acoustic line signal then undergoing processing such as envelope detection, logarithmic compression, and the like, converting it into a luminance signal, the luminance signal then undergoing coordinate transformation to an orthogonal coordinate system. The display controller 113 causes the display 114 to display a B mode image in which tissue of a subject is drawn.

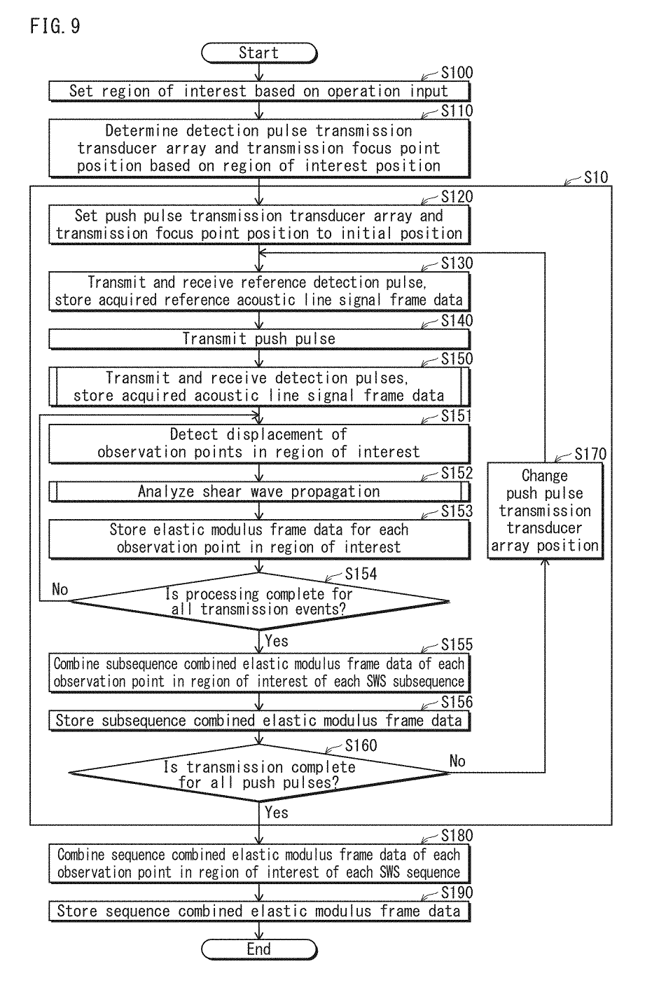

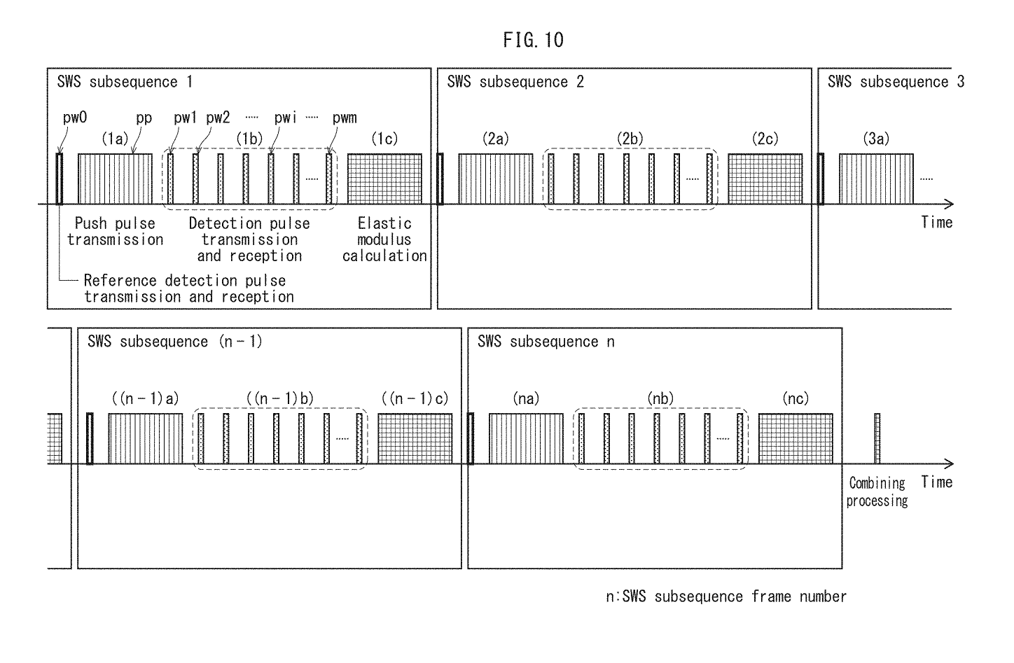

FIG. 9 is a flowchart illustrating an operation of ultrasound elastic modulus calculation of the ultrasonic diagnostic device 100. FIG. 10 is a schematic diagram illustrating an outline of an SWS sequence process in the ultrasonic diagnostic device 100.

[Steps S100 to S140]

In step S100, in a state in which a B mode image, which is a tomographic image of a subject acquired in real time by the probe 101 is displayed on the display 114, the region of interest setter 103 inputs information designated by a user via the operation input unit 102 in order to set a region of interest roi representing an analysis target range in the subject using position of the probe 101 as a reference, and outputs the region of interest roi to the controller 112.

Designation of a region of interest roi by a user is performed, for example, by display on the display 114 of a latest B mode image stored in the data storage 111, and designating the region of interest roi via an input unit (not illustrated) such as a touch panel, mouse, or trackball. Designation of a region of interest roi is not limited to this example, and, for example, an entire region of a B mode image may be a region of interest roi, or a fixed range including a central portion of the B mode image may be a region of interest roi. Further, when designating a region of interest roi, a tomographic image may be acquired.

In step S110, the detection pulse generator 105 inputs information indicating the region of interest roi from the controller 112, and, according to a method illustrated in FIG. 4 and described above, sets position of a transmission focus point F of a detection pulse and a detection pulse transmission transducer array Tx such that an ultrasonic beam converges at a position outside the region of interest roi and passes through the entirety of the region of interest roi. Information indicating position of the transmission focus point F and the detection pulse transmission transducer array Tx is outputted to the transmission beamformer 106 as a transmission control signal together with pulse width of a detection pulse.

In step S120, the push pulse generator 104 sets push pulse transmission focus point F position and push pulse transmission transducer array Px according to initial conditions. The push pulse generator 104 inputs information indicating the region of interest roi from the controller 112, and sets the push pulse transmission focus point F position and the push pulse transmission transducer array Px as described above, such that an ultrasonic beam converges at a defined position in the region of interest roi. Alternatively, a defined position outside the region of interest roi that is near the region of interest roi and allows a shear wave to arrive at the region of interest roi may be set as a transmission focus point F. According to the present example, as illustrated in FIG. 3B, a plurality of push pulses are generated in an entire SWS sequence. In this case, among transmission focus point F positions, for each SWS subsequence, array direction transmission focus point positions fx1, fx2 coincide with positions internally dividing a region of interest roi in the array direction. In a first SWS subsequence, array direction transmission focus point position fx1 is adopted. Depth direction transmission focus point position fz coincides with depth d to a region of interest roi center, and the push pulse transmission transducer array Px is set to be all of the transducers 101a.

Information indicating position of transmission focus point F and the push pulse transmission transducer array Px is outputted to the transmission beamformer 106 as a transmission control signal together with pulse width of a push pulse.

In step S130, the transmission beamformer 106 causes transducers included in a detection pulse transmission transducer array Tx to transmit a detection pulse pw0 to the region of interest roi in the subject, and the reception beamformer 108 receives reflected waves ec of a detection pulse to generate reference acoustic line signal frame data ds0 as a reference for tissue displacement. The reference acoustic line signal frame data ds0 is outputted to and stored by the data storage 111. Acoustic line signal frame data is described later.

In step S140, the transmission beamformer 106 causes transformers included in the push pulse transmission transducer array Px to transmit a push pulse pp that converges to a specific site in the subject. More specifically, the transmission beamformer 106 generates a transmission profile based on a transmission control signal including information indicating position of the transmission focus point F, the push wave transmission transducer array Px, and push pulse width acquired by the push pulse generator 104. The transmission profile includes pulse signal sp and delay time tpl with respect to each transmission transducer included in the push pulse transmission transducer array Px. Thus, a transmission signal scl is supplied to each transmission transducer based on the transmission profile. Each transmission transducer transmits a push pulse pp in a pulse that converges to the specific site in the subject.

Here, in the first SWS subsequence, when transmitting a push pulse, the transmission beamformer 106 generates an initial transmission profile based on the transmission control signal set in step S120. In the second SWS subsequence, when transmitting a push pulse, the transmission beamformer 106 generates a transmission profile based on a transmission control signal changed in step S170.

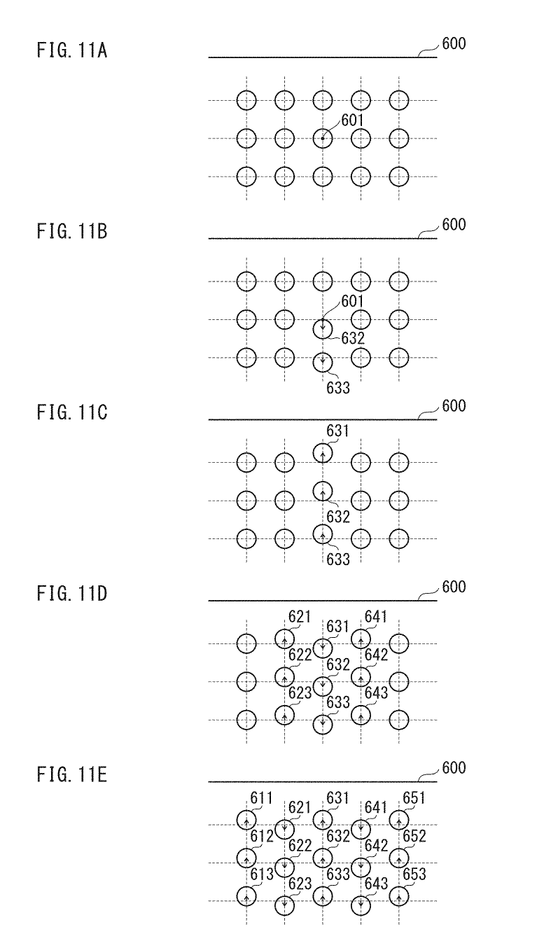

Here, generation of a shear wave by a push pulse pp is described with reference to schematic diagrams FIG. 11A to FIG. 11E. FIG. 11A to FIG. 11E are schematic diagrams showing generation and propagation of a shear wave. FIG. 11A is a schematic diagram illustrating tissue prior to application of a push pulse in a region in a subject corresponding to a region of interest roi. In FIG. 11A to FIG. 11E, each circle “0” indicates a portion of tissue in a subject in a region of interest roi, intersections of dashed lines indicating center positions of circles “0” when tissue is not under load.

Here, when the probe 101, in close contact with a skin surface 600, applies a push pulse pp to a transmission focus point 601 as the specific site, tissue 632 at the transmission focus point 601 is pushed and moved in a travel direction of the push pulse pp, as illustrated in the schematic diagram of FIG. 11B. Further, tissue 633, which is in the travel direction of the push pulse pp from the tissue 632, is pushed by the tissue 632 and moves in the travel direction of the push pulse.

Subsequently, when transmission of the push pulse pp ends, the tissue 632, 633 attempts to return to original positions, and therefore tissue 631, the tissue 632, and the tissue 633 start vibrating along the travel direction of the push pulse, as illustrated in the schematic diagram of FIG. 11C.

As illustrated in the schematic diagram of FIG. 11D, vibrations propagate to tissue 621, 622, 623 and tissue 641, 642, 643, which are adjacent to the tissue 631, 632, 633.

Further, as illustrated in the schematic diagram of FIG. 11E, vibrations further propagate to tissue 611, 612, 613 and tissue 651, 652, 653. Accordingly, in the subject, vibrations propagate in a direction perpendicular to the direction of vibration. In other words, a shear wave is generated at a point of application of the push pulse pp, and propagates in the subject.

[Step S150]

Description continues with reference to FIG. 9.

In step S150, detection pulse pwi is repeatedly transmitted and received with respect to the region of interest roi, and an acquired acoustic line signal frame data dsi sequence is stored. More specifically, the transmission beamformer 106 causes transducers included in the detection pulse transmission transducer array Tx to transmit the detection pulse pwi with respect to the region of interest roi in the subject, and the reception beamformer 108 generates acoustic line signal frame data dsi based on detection pulse reflected waves ec received by transducers included in a detection pulse reception transducer array Rx. Immediately after transmission of push pulse pp ends, the above process is repeated 10,000 times per second, for example. Thus, immediately after shear wave generation and until propagation ends, an acoustic line signal frame data dsi tomographic image for the region of interest roi of the subject is repeatedly generated. A generated acoustic line signal frame data dsi sequence is outputted to and stored by the data storage 111.

A method of generating acoustic line signal frame data dsi in step S150 is described in detail later.

[Step S151]

In step S151, the displacement detector 109 detects displacement of an observation point Pij in the region of interest roi for each transmission event.

More specifically, the displacement detector 109 acquires reference acoustic line signal frame data ds0 stored in the data storage 111 in step S130. As above, reference acoustic line signal frame data ds0 is acoustic line signal frame data dsi acquired prior to push pulse pp transmission, that is, prior to shear wave generation.

Next, the displacement detector 109 detects displacement of pixels at a time of acquisition of the acoustic line signal frame data dsi, from differences in the acoustic line signal frame data dsi stored in the data storage 111 in step S150 from the reference acoustic line signal frame data ds0. More specifically, for example, the acoustic line signal frame data dsi is divided into regions of defined size such as eight pixels by eight pixels, each region is pattern matched to the reference acoustic line signal frame data ds0, and displacement of each pixel of the acoustic line signal frame data dsi is thereby detected.

As a method of pattern matching, an example method can be used in which, for each region, a total value is calculated that is a sum of absolute values of difference in luminance calculated for each pixel with respect to a reference region of the same size in the reference acoustic line signal frame data ds0, then a combination of a region and the reference region that has a lowest total value is assumed to be the same region, and a distance between a reference point (for example, a top-left corner) of the region and a reference point of the reference region is calculated as displacement.

Size of a region may be a size other than eight pixels by eight pixels, and instead of a total value of absolute values of luminance differences a total value of squared luminance differences may be used. Further, as displacement, a difference in y coordinates (difference in depth) may be calculated between a reference point of a region and a reference point of a reference region. Thus, with respect to tissue of a subject corresponding to observation points Pij of each acoustic line signal frame data dsi, how far the tissue moves due to a push pulse or shear wave is calculated as displacement.

Note that method of displacement detection is not limited to pattern matching. Any technique may be used for detecting motion between two sets of acoustic line signal frame data dsi, such as correlation processing between acoustic line signal frame data dsi and reference acoustic line signal frame data ds0, for example. The displacement detector 109 generates each frame data displacement by associating displacement of each observation point pertaining to one frame of acoustic line signal frame data dsi with coordinates of the observation point, and outputting a generated displacement frame data pti sequence to the data storage 111.

[Step S152]

In step S152, the propagation analyzer 1101 detects a wavefront from the displacement frame data pti of an observation point Pij in the region of interest roi in each transmission event.

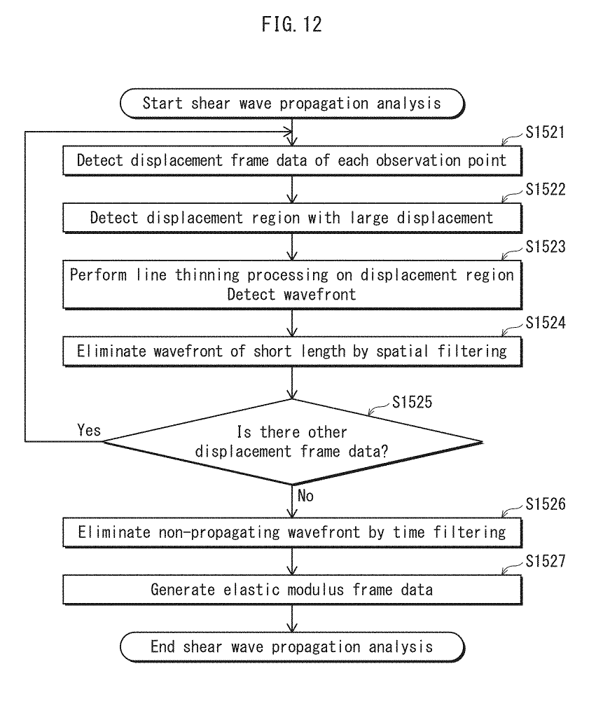

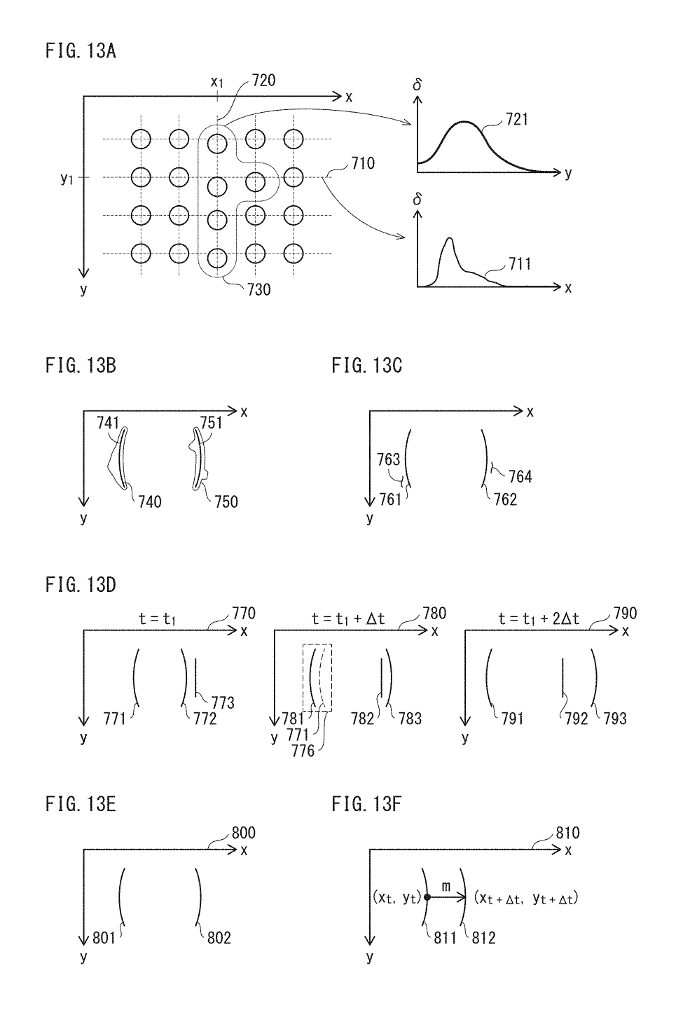

Details are described with reference to the flowchart of FIG. 12. FIG. 12 is a flowchart illustrating a shear wave propagation information analysis operation. FIG. 13A to FIG. 13F are schematic diagrams showing a shear wave propagation analysis operation.

Initially, displacement frame data pti for each observation point Pij corresponding to a transmission event is acquired from the data storage 111 (step S1521).

Next, a displacement region of relatively large displacement is extracted (step S1522). The propagation analyzer 1101 extracts a displacement region from displacement frame data pti in which displacement is greater than a defined threshold.

The following description references FIGS. 13A, 13B, 13C, 13D, 13E, and 13F.

FIG. 13A illustrates an example of a displacement image represented by displacement frame data. As in FIG. 11A to 11E, each circle “◯” in the drawing indicates a portion of tissue in a subject corresponding to a region of interest roi, a position of which is centered on an intersection of dashed lines prior to application of a push pulse. Further, the x axis is the array direction of transducers of the probe 101 and the y axis is the depth direction of a subject. The propagation analyzer 1101 treats a displacement δ for each y coordinate as a function of coordinates x, and extracts a region for which displacement δ is large, by using a dynamic threshold. Further, treating displacement δ for each x coordinate as a function of coordinates y, and using a dynamic threshold, a region that exceeds a threshold is extracted as a region for which displacement δ is large. Here, a dynamic threshold is determined by performing signal analysis or image analysis of a target region. The threshold is not a fixed value, but varies according to factors such as width and maximum value of a signal of a target region. FIG. 13A illustrates a graph 711 in which displacement is plotted on a straight line 710 for which y=y₁, and a graph 721 in which displacement is plotted on a straight line 720 for which x=x₁. Thus, for example, a displacement region 730 in which displacement δ is greater than a threshold can be extracted.

Next, the propagation analyzer 1101 extracts a wavefront by performing thinning processing on the displacement region (step S1523). Displacement regions 740, 750 shown in the schematic diagram of FIG. 13B are each a region extracted as the displacement region 730 in step S1522. The propagation analyzer 1101 extracts a wavefront by using, for example, a Hilditch thinning algorithm. For example, in the schematic diagram of FIG. 13B, a wavefront 741 is extracted from the displacement region 740 and a wavefront 751 is extracted from the displacement region 750. Note that the thinning algorithm is not limited to Hilditch, and any thinning algorithm may be used. Further, for each displacement region, a process of removing coordinates for which displacement δ is equal to or less than a threshold may be repeated while increasing the threshold until width of the displacement region is a single pixel. The propagation analyzer 1101 outputs an extracted wavefront as wavefront frame data wfi to the data storage 111.

Next, the propagation analyzer 1101 performs spatial filtering with respect to wavefront frame data wfi, eliminating wavefronts of short length (step S1524). For example, length of each wavefront extracted in step S1523 is extracted, and a wavefront that has a length less than half of the average length of all wavefronts is eliminated as noise. More specifically, as indicated in the wavefront image of FIG. 13C, the propagation analyzer 1101 calculates the average length of wavefronts 761, 762, 763, 764, and eliminates as noise the wavefronts 763 and 764, which each have a shorter length than the average length. Thus, erroneously detected wavefronts can be eliminated.

The propagation analyzer 1101 performs the operations of steps S1521 to S1524 with respect to all displacement frame data pti (step S1525). Thus, wavefront frame data wfi is generated one-to-one with respect to displacement frame data pti.

Next, the propagation analyzer 1101 performs time filtering with respect to wavefront frame data wfi, eliminating wavefronts that aren't propagating (step S1526). More specifically, with respect to two or more temporally consecutive wavefront frame data wfi, a change over time in wavefront position is detected, and a wavefront for which speed is abnormal is eliminated as noise.

The propagation analyzer 1101, for example, detects wavefront position changes over time between wavefront image 770 at time t=t₁, wavefront image 780 at time t=t₁+Δt, and wavefront image 790 at time t=t₁+2Δt. For example, with respect to wavefront 771, in the wavefront image 780, the propagation analyzer 1101 performs correlation processing in a region 776 centered on the same position as the wavefront 771, the region 776 being an area in which a shear wave could possibly move in the time period Δt in directions perpendicular to the wavefront (the x axis direction in FIG. 13D). Thus, the correlation processing is performed in a range that includes both a positive x axis direction (right in FIG. 13D) and a negative x axis direction (left in FIG. 13D) from the wavefront 771. This is to detect both transmitted and reflected waves. Thus, the propagation analyzer 1101 detects that a movement destination of the wavefront 771 is a wavefront 781 in the wavefront image 780, and calculates a movement distance of the wavefront 771 over the time period Δt. In the same way, with respect to wavefronts 772 and 773, in the wavefront image 780, the propagation analyzer 1101 performs correlation processing in regions centered on the same positions as each wavefront that represent areas in which a shear wave could possibly move in the time period Δt in directions perpendicular to the wavefronts. Thus, the propagation analyzer 1101 detects that the wavefront 772 moves to a position of a wavefront 783 and the wavefront 773 moves to a position of a wavefront 782.

The same processing is performed for the wavefront image 780 and the wavefront image 790, detecting movement of wavefront 781 to a position of wavefront 791, wavefront 782 to a position of wavefront 792, and wavefront 783 to a position of wavefront 793. Here, the single wavefront indicated by the wavefront 773, the wavefront 782, and the wavefront 792 has a significantly shorter travel distance than other wavefronts (a significantly slower propagation speed). Such a wavefront is most likely a false positive, and is therefore eliminated as noise. Thus, as shown in wavefront frame data 300 of FIG. 13E, wavefronts 801 and 802 can be detected.

According to these operations, a sequence of wavefront frame data wfi over time can be generated. The propagation analyzer 1101 outputs a generated wavefront frame data wfi sequence to the data storage 111. At this time, generated wavefront correspondence information may be outputted to the data storage 111. Here, wavefront correspondence information means information indicating which wavefront in each wavefront image a given wavefront corresponds to. For example, when it is detected that the wavefront 772 moves to the position of the wavefront 783, the information indicates that the wavefront 783 and the wavefront 772 are the same wavefront.

Next, the propagation analyzer 1101 generates an elastic modulus frame data eli sequence (step S1527). More specifically, the propagation analyzer 1101 detects wavefront position and speed at each time, from the wavefront frame data wfi at each time and the wavefront correspondence information. Further, from the relationship between wavefront frame data wfi and tomographic images, the propagation analyzer 1101 calculates elastic modulus from maximum speed of a shear wave in a plurality of wavefront frame data for each pixel of a tomographic image, and generates an elastic modulus frame data eli sequence associating each pixel of tomographic images with elastic modulus.

Generation of elastic modulus frame data eli is described with reference to FIG. 13E. FIG. 13E shows wavefront frame data wfi at a time t and wavefront frame data wfi at a time t+Δt combined as wavefront frame data 810. Here, it is assumed that correspondence information exists that indicates that a wavefront 811 at time t and a wavefront 812 at time t+Δt are the same wavefront. The propagation analyzer 1101 detects, from correspondence information, coordinates (x_{t+Δt}, y_{t+Δt}) on the wavefront 812 corresponding to coordinates (xₜ, yₜ) on the wavefront 811. Thus, the propagation analyzer 1101 can estimate that a shear wave that passes through coordinates (xₜ, yₜ) at time t arrives at coordinates (x_{t+Δt}, y_{t+Δt}) at time t+Δt. Accordingly, velocity v(xₜ, yₜ) of the shear wave that passed through coordinates (xₜ, yₜ) can be estimated as a value obtained by dividing a distance m between coordinates (xₜ, yₜ) and coordinates (x_{t+Δt}, y_{t+Δt}) by a required time Δt. That is:

v(xₜ,yₜ)=m/Δt=√{(x_{t+Δt}−xₜ)²+(y_{t+Δt}−yₜ)²}/Δt

The propagation analyzer 1101 performs the processing above with respect to all wavefronts, acquires shear wave velocity for all coordinates through which a wavefront passes, and calculates elastic modulus at each coordinate based on shear wave speed. Elastic modulus is proportional to the square of speed of a shear wave, and is calculated based on the formula below.

el(xₜ,yₜ)=K×v(xₜ,yₜ)²

K is a constant, and for human body tissue is approximately three. This completes shear wave propagation analysis.

[Steps S153 to S190]

Description continues with reference to FIG. 9. The propagation analyzer 1101 outputs a generated elastic modulus frame data efi sequence to the data storage 111 (step S153). It is determined whether or not the processing of steps S151 to S153 is complete for all specified transmission events (step S154). If not complete, processing returns to step S151 and processing is performed for the next detection pulse transmission event. If complete, processing proceeds to step S155.

Next, the combiner 1102 combines elastic modulus frame data eli of shear waves corresponding to a plurality of transmission events included in a SWS subsequence with observation points Pij as reference, calculates SWS subsequence combined elastic modulus frame data emk corresponding to the SWS subsequence (step S155), stores same in the data storage 111 (step S156), and processing proceeds to step S160. Simultaneously or alternatively, SWS subsequence combined shear wave propagation speed frame data corresponding to the SWS subsequence may be calculated.

In step S160, it is determined whether or not the processing of steps S130 to S153 is complete for all designated push pulses (step S160). If not complete, processing proceeds to step S170.

In step S170, the push pulse generator 104 changes the push pulse transmission focus point F position and the push pulse transmission transducer array Px. According to the present example, as described with reference to FIG. 8, a SWS sequence is generated from a plurality (n) of push pulses. At this time, among transmission focus point F positions, the transmission focus point F array direction transmission focus point positions fx, as illustrated in FIG. 3B, coincide with positions internally dividing the array direction of a region of interest roi for each SWS subsequence, causing generation of a plurality of push pulses in an entire SWS sequence. For example, as illustrated in FIG. 3B, when n is 2, in the second SWS subsequence, fx2 in FIG. 3B is adopted as the array direction transmission focus point position fx. Depth direction transmission focus point position fz coincides with depth d to a region of interest roi center, and the push pulse transmission transducer array Px is set to be all of the transducers 101a.

Information indicating the transmission focus point F position and the push pulse transmission transducer array Px is outputted to the transmission beamformer 106 as a transmission control signal together with pulse width of a push pulse, and processing proceeds to step S130.

In step S160, if it is determined that processing is complete for all designated push pulses, processing proceeds to step S180.

In step S180, the subsequence combiner 1103 combines SWS subsequence combined elastic modulus frame data emk of SWS subsequences included in the SWS sequence with observation points Pij as reference, calculates SWS sequence combined elastic modulus frame data elm corresponding to the SWS sequence (step S180), and stores same in the data storage 111 (step S190). Simultaneously or alternatively, SWS sequence combined shear wave propagation speed frame data corresponding to the SWS sequence may be calculated.

This completes SWS sequence processing shown in FIG. 9. According to the ultrasound elastic modulus measurement processing above, SWS sequence combined elastic modulus frame data elm can be calculated.

3. Details of Processing in Step S150

The following describes details of acoustic line signal frame data dsi generation processing by reception beamforming in step S150.

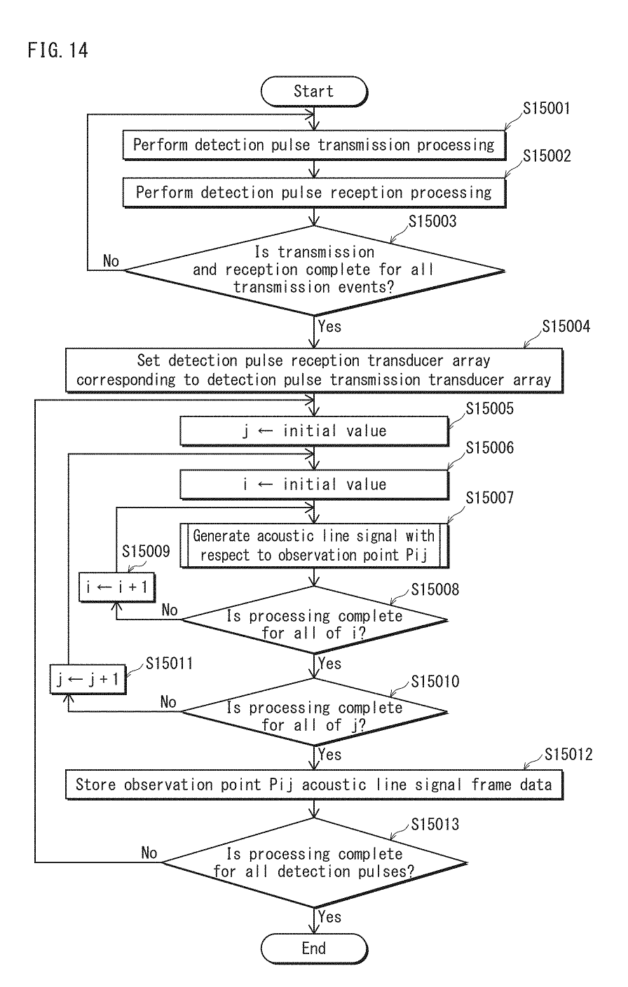

FIG. 14 is a flowchart illustrating a beamforming operation of the reception beamformer 108.

First, in step S15001, the transmission beamformer 106 performs transmission processing (a transmission event) supplying a transmission signal for causing each transducer included in the detection pulse transmission transducer array Tx among the transducers 101a of the probe 101 to transmit an ultrasonic beam.

Next, in step S15002, the reception beamformer 108 generates a reception signal based on an electrical signal obtained from reception of reflected ultrasound by the probe 101 and outputs same to the data storage 111, storing the reception signal in the data storage 111. It is then determined whether or not ultrasound transmission and reception is complete for all of a number of designated transmission events (step S15003). If not complete, processing returns to step S15001, and a transmission event from the detection pulse transmission transducer array Tx is performed, and if complete, processing proceeds to step S15004.

Next, in step S15004, the controller 112 matches an array center corresponding to a transmission event to an array center of the detection pulse transmission transducer array Tx, and selects reception transducers Rpl at least including the transducers included in the detection pulse transmission transducer array Tx to set a detection pulse reception transducer array Rx.

Next, coordinates ij indicating a position of an observation point Pij in a region of interest roi are initialized to minimum values (steps S15005, S15006), and an acoustic line signal is generated for the observation point Pij (step S15007). Details of processing in step S15007 are provided later.

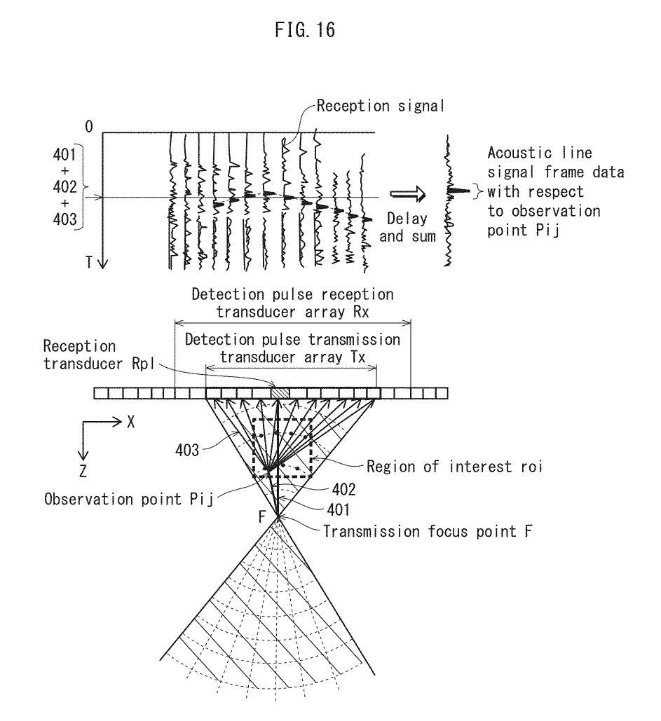

Next, by incrementing coordinates ij and repeating step S15007, acoustic line signals are generated for all observation points Pij (dots “•” in FIG. 16) positioned at coordinates ij in the region of interest roi. It is determined whether generation of acoustic line signals is complete for all observation points Pij in the region of interest roi (steps S15008, S15010). If not complete, coordinates ij are incremented (steps S15009, S15011) and an acoustic line signal is generated for an observation point Pij (step S15007). If complete, processing proceeds to step S15012. At this stage, acoustic line signal frame data dsi has been generated for all observation points Pij in the region of interest roi associated with one transmission event, and outputted to and stored by the data storage 111.

Next, it is determined whether or not acoustic line signal generation is complete for detection pulses for all transmission events (step S15013). If not complete, processing returns to step S15005, and acoustic line signal generation based on the detection pulse of the next transmission event is performed (steps S15005 to S15012). If complete, processing ends.

Thus, processing of step S150 in FIG. 9 is completed.

4. Details of Processing in Step S15007

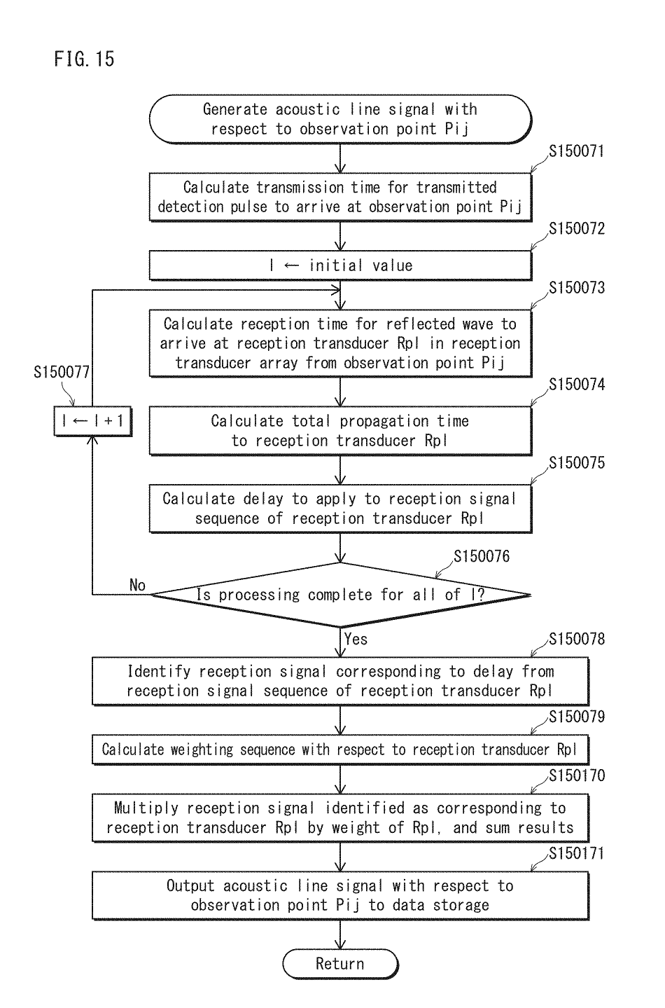

The following describes an acoustic line signal generation processing operation for an observation point Pij in step S15007. FIG. 15 is a flowchart illustrating an acoustic line signal generation operation for an observation point Pij by the detection beamformer 108. FIG. 16 is a schematic diagram for describing the acoustic line signal generation operation for an observation point Pij by the reception beamformer 108.

Initially, in step S150071, the delay processing section 10831 calculates, for any observation point Pij present in a region of interest roi, a transmission time for transmitted ultrasound to arrive at an observation point Pij in the subject. As described above, transmission time can be calculated by first calculating the transmission path 404 from a reception transducer Rpl in the detection pulse reception transducer array Rx to the observation point Pij as a difference between a first path 401 from an array center of the detection pulse reception transducer array Rx to the transmission focus point F and a second path 402 from the transmission focus point F and the observation point Pij (401-402), and dividing transmission path length by ultrasound speed cs.

Next, an identification number 1 of reception transducers Rpl in the detection pulse reception transducer array Rx obtained from the detection pulse reception transducer array Rx is initialized to a minimum value of the detection pulse reception transducer array Rx (step S150072), and reception time at which ultrasound transmitted in a subject and reflected at an observation point Pij arrives at the reception transducer Rpl of the detection pulse reception transducer array Rx is calculated (step S150073). Reception time can be calculated by dividing a geometrically determined length of the path 403 from the observation point Pij to the reception transducer Rpl by ultrasound speed cs. Further, from a sum of transmission time and reception time, total propagation time for ultrasound transmitted from the detection pulse transmission transducer array Tx and reflected at an observation point Pij to arrive at a reception transducer Rpl is calculated (step S150074), and delay for each reception transducer Rpl is calculated from differences in total propagation time for each reception transducer Rpl in the detection pulse reception transducer array Rx (step S150075).

It is then determined whether or not calculation of delay is complete for all reception transducers Rpl in the detection pulse reception transducer array Rx (step S150076). If not complete, the coordinate 1 is incremented (step S150077), and delay is calculated for another reception transducer Rpl (step S150073). If complete, processing proceeds to step S150078. At this stage, reflected ultrasound arrival delay for an observation point Pij for all reception transducers Rpl in the detection pulse reception transducer array Rx has been calculated.

In step S150078, from a sequence of reception signals corresponding to reception transducers Rpl in the detection pulse reception transducer array Rx, the delay processing section 10831 identifies a reception signal corresponding to a time obtained by subtracting delay from each reception transducer Rpl as a reception signal based on ultrasonic reflected from an observation point Pij.

Next, a weighting calculator (not illustrated) calculates a weight sequence for each reception transducer Rpl such that a transducer positioned at a center in the array direction of the detection pulse reception transducer array Rx has a maximum weight (step S150079). The summing section 10832 multiplies reception signals identified as corresponding to reception transducers Rpl by weights of the reception transducers Rpl, and sums the results to generate an acoustic line signal corresponding to an observation point Pij (step S150170). An acoustic line signal generated for an observation point Pij is outputted to and stored by the data storage 111 (step S150171).

Thus, processing of step S15007 in FIG. 14 is completed.

<Evaluation Test>

1. Acoustic Pressure of Acoustic Line Signal Obtained by Detection Pulse Transmission and Reception

Evaluation is performed regarding acoustic pressure of acoustic line signals generated by detection pulse transmission and reception pertaining to the ultrasonic diagnostic device 100.

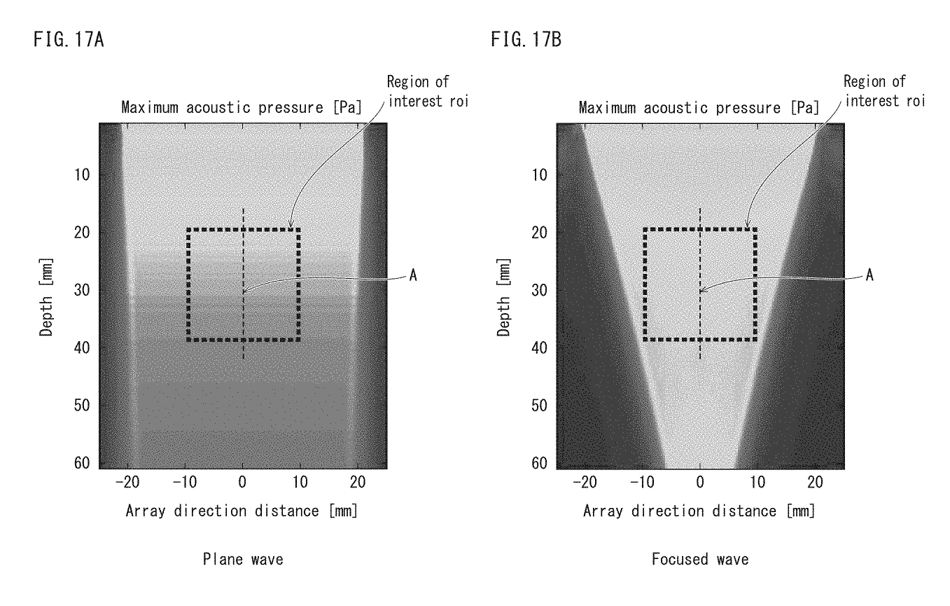

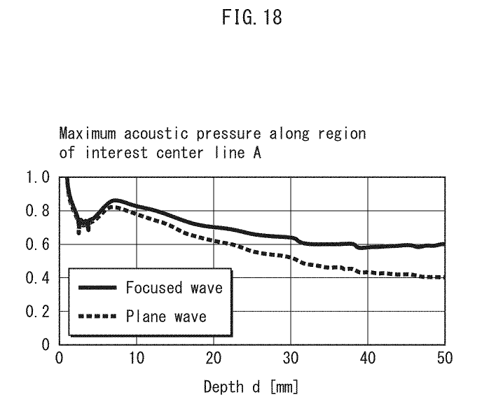

FIG. 17A and FIG. 17B are simulation images indicating maximum sound pressure of acoustic line signals generated based on detection pulses. FIG. 17A is an image pertaining to a comparative example using a plane wave pulse as a detection pulse, and FIG. 17B is an image pertaining to a working example using a converging wave as a detection pulse pertaining to the ultrasonic diagnostic device 100. FIG. 18 illustrates results of maximum sound pressure of acoustic line signals on a center axis A of the region of interest roi in FIG. 17A and FIG. 17B; the broken line being results of the comparative example and the unbroken line being results of the working example of the ultrasonic diagnostic device 100. As illustrated in FIG. 17A, 17B, and FIG. 18, at a subject depth of approximately 5 mm and greater, maximum acoustic pressure of an acoustic line signal for the working example using a converging detection pulse is in a range at most approximately 1.5 times larger than that of the comparative example using a plane wave detection pulse. This is thought to be because ultrasonic beam energy density of a detection pulse in a region of interest roi is high in inverse proportion to an irradiated area, and therefore higher in the working example using a converging wave than in the comparative example using a plane wave. Based on such acoustic line signal frame data, calculated elastic modulus frame data, SWS subsequence combined elastic modulus frame data, and SWS sequence combined elastic modulus frame data have higher S/N ratios according to the working example than according to the comparative example.

<Effects>

The ultrasonic diagnostic device 100 pertaining to Embodiment 1 as described above includes the push pulse generator 104 that sets a specific site in a subject and causes a plurality of the transducers 101a to transmit a push pulse pp; the detection pulse generator 105 that, following the push pulse pp, causes multiple transmissions of a detection pulse pwi that converges outside a region of interest roi in the subject and passes through the region of interest roi; the displacement detector 109 that generates an acoustic line signal for each of a plurality of observation points Pij in the region of interest roi in response to the detection pulses pwi in order to detect displacement of tissue in the region of interest roi from an acoustic line signal frame data dsi sequence; and the elastic modulus calculator 110 that generates a wavefront frame data wfi sequence representing shear wave wavefront position, and calculates shear wave propagation speed and/or elastic modulus frame data emk in the region of interest roi based on the wavefront frame data wfi sequence.

According to this configuration, in ultrasound elastic modulus measurement, signal acquisition time resolution and signal S/N for elasticity image generation can be improved over conventional art using a plane wave detection pulse.

In ultrasound elastic modulus measurement according to a SWS sequence, in order to improve signal S/N, a region of interest including a site of elastic modulus measurement is usually set near or around a push pulse transmission focus point. According to the configuration pertaining to the embodiment, by adopting a configuration in which a detection pulse pwi that converges outside a region of interest roi in a subject and passes through the region of interest roi is transmitted and reflected detection waves are received, it is possible for the detection pulse to irradiate a region of interest including a site of elastic modulus measurement without excess or shortfall, and to calculate elastic modulus based on reception thereof. Thus, ultrasonic beam energy density of a detection pulse in a region of interest can be increased, and obtained signal acquisition time resolution and signal S/N for elasticity image generation can be improved. Further, processing load for one transmission event until elastic modulus calculation can be reduced, and signal acquisition time resolution can be improved.

### Embodiment 2

According to the ultrasonic diagnostic device 100 pertaining to Embodiment 1, as illustrated in FIG. 4, the detection pulse generator 105 sets all of a plurality of the transducers 101a as the detection pulse transmission transducer array Tx, sets transmission pocus point F position such that array direction transmission focus point position fx coincides with an array direction center position of a region of interest roi, sets depth direction transmission focus point position fz1 such that an ultrasonic beam passes through the entire region of interest roi, and causes the plurality of the transducers 101a to transmit a detection pulse. Further, as illustrated in FIG. 8, in all SWS subsequences (1 to n) that constitute a SWS sequence, transmission focus point F position and the detection pulse transmission transducer array Tx are unchanging.

However, detection pulse configuration is not limited to the above configuration, and transmission focus point F position and detection pulse transmission transducers array Tx configuration may be changed as appropriate, as long as the ultrasonic beam converges to a transmission focus point F positioned outside a region of interest roi in a subject, and the ultrasonic beam passes through the region of interest roi.

The ultrasonic diagnostic device 100A pertaining to Embodiment 2 is different from Embodiment 1 in that transmission position of a detection pulse pwi is gradually moved in the array direction for each subsequence along with gradual movement of a push pulse pp convergence site, repeatedly transmitting and receiving the detection pulse pwi with respect to partial regions of a region of interest roi, and elastic modulus emk (k=1 to n) calculated for a partial region of the region of interest roi for each subsequence is combined to calculate SWS sequence combined elastic modulus elm for the entire region of interest roi.

The following describes the ultrasonic diagnostic device 100A.

<Configuration>

According to the ultrasonic diagnostic device 100A, configuration of a detection pulse generated by the detection pulse generator 105 is different from the configuration of Embodiment 1, and therefore the following describes detection pulse configuration pertaining to the ultrasonic diagnostic device 100A. Configuration other than that of the detection pulse is the same as for the ultrasonic diagnostic device 100 and is not described here.

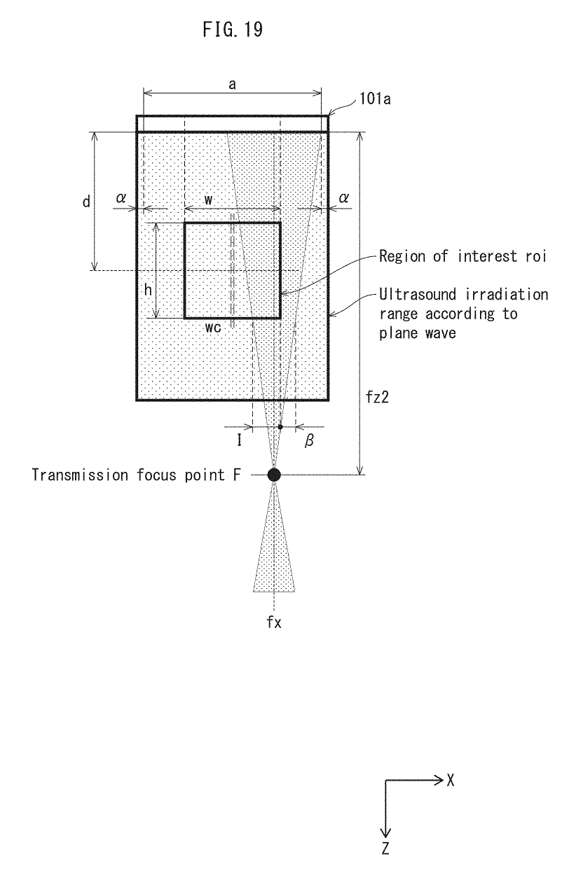

FIG. 19 is a schematic diagram illustrating a schematic configuration of a detection pulse generated by the detection pulse generator 105 in the ultrasonic diagnostic device 100A pertaining to Embodiment 2. As illustrated in FIG. 19, according to the ultrasonic diagnostic device 100A, the detection pulse generator 105 sets a depth direction transmission focus point position of a detection pulse such that an ultrasonic beam converges at a transmission focus point F that is outside a region of interest roi and a position deeper than the region of interest roi and the depth direction transmission focus point position is at a depth fz2 such that the ultrasonic beam passes through a portion of the region of interest roi. Further, the detection pulse transmission transducer array Tx is a portion of the transducers 101a. Further, among transmission focus point F positions, it suffices that array direction transmission focus point position fx is set such that an ultrasonic beam at least partially overlaps the region of interest roi.

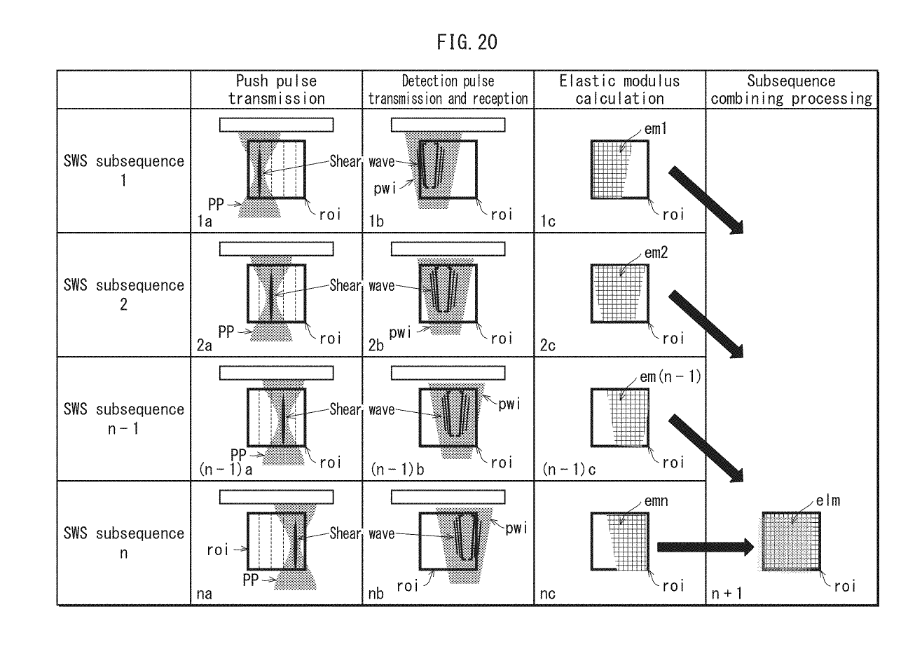

FIG. 20 is a schematic diagram illustrating an outline of SWS sequence processing composed from SWS subsequences in the ultrasonic diagnostic device 100A. Tissue elastic modulus measurement by the ultrasonic diagnostic device 100A is configured from a SWS sequence including a plurality (n) of SWS subsequences.

The SWS subsequences (1 to n) are composed from processing including: push pulse transmission to transmit a push pulse pp in a subject to excite a shear wave and gradually moving a specific site at which a push pulse pp converges in the array direction for each subsequence; multiple repetitions of detection pulse transmission and reception to transmit and receive detection pulse pwi with respect to a partial region of a region of interest roi, gradually moving a transmission position in the array direction for each subsequence, similar to the movement of the push pulse pp; and elastic modulus calculation of performing shear wave propagation analysis with respect to each partial region of the region of interest roi and calculating shear wave propagation speed and elastic modulus emk (k=1 to n).

In a SWS sequence, after a plurality of SWS subsequences (1 to n) are obtained, elastic modulus frame data emk calculated with respect to each partial region of the region of interest roi for each SWS subsequence is combined by subsequence combining processing to calculate SWS sequence combined elastic modulus frame data elm with respect to the entire region of interest roi.

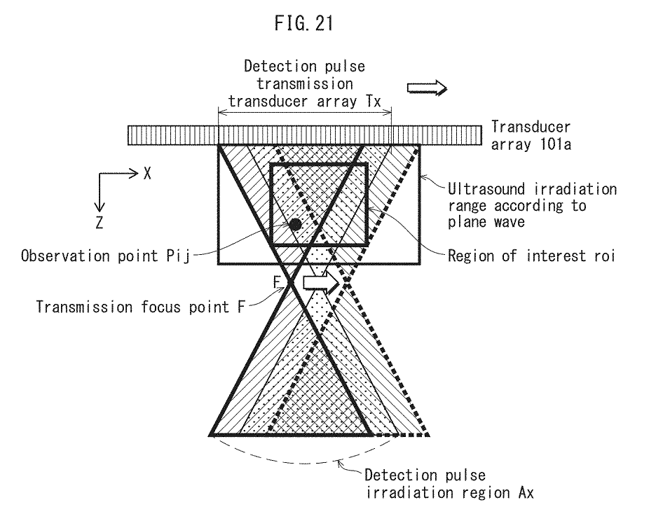

FIG. 21 is a schematic diagram illustrating an outline of a reception beamforming method of the ultrasonic diagnostic device 100A. According to the ultrasonic diagnostic device 100A, the subsequence combiner 1103 uses position of an observation point Pij as an index to sum subsequence combined elastic modulus frame data emk of a shear wave calculated with respect to a partial region of a region of interest roi corresponding to a plurality of SWS subsequences, and thereby calculates SWS sequence combined elastic modulus frame data emk with respect to the entire region of interest roi corresponding to an SWS sequence.

<Operations>

The following describes operations of a SWS sequence of the ultrasonic diagnostic device 100A.

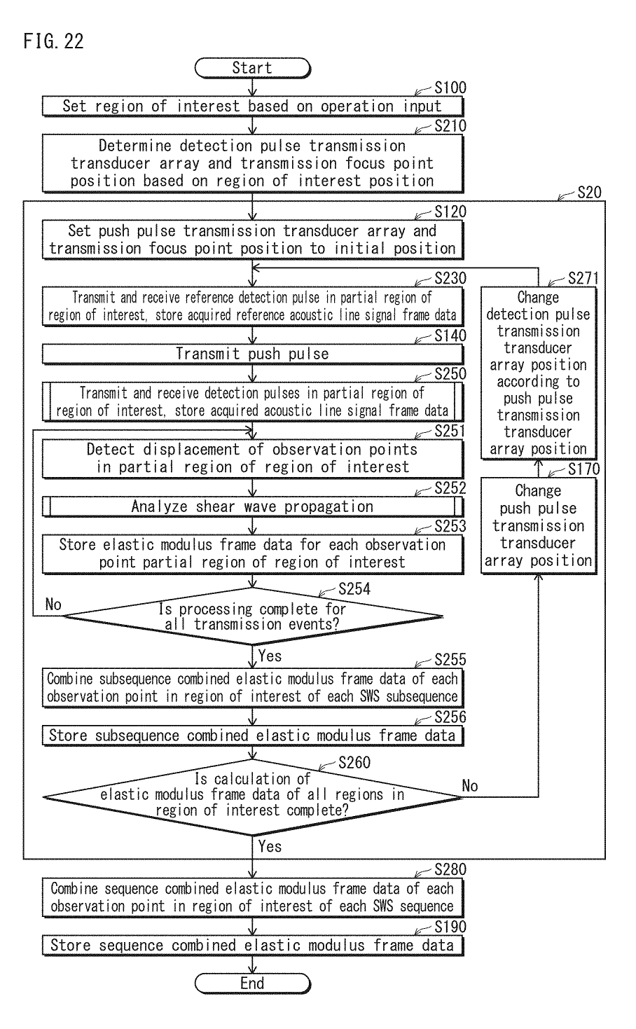

FIG. 22 is a flowchart illustrating an ultrasound elastic modulus calculation operation of the ultrasonic diagnostic device 100A. Processing that is the same as that of the ultrasonic diagnostic device 100 in FIG. 9 is denoted by the same reference signs and only outlined, while only steps including different processing are described below.

In step S100, the region of interest setter 103 inputs information designated by a user, sets a region of interest roi with reference to position of the probe 101, and outputs to the controller 112.

In step S210, the detection pulse generator 105 inputs information indicating the region of interest roi from the controller 112, and, according to a method illustrated in FIG. 19 and described above, sets a transmission focus point F position of a detection pulse and the detection pulse transmission transducer array Tx such that an ultrasonic beam converges at a position outside the region of interest roi and passes through a partial region of the region of interest roi. Information indicating transmission focus point F position and detection pulse transmission transducer array Tx is outputted to the transmission beamformer 106 as a transmission control signal together with pulse width of a detection pulse.

In step S120, the push pulse generator 104 sets the push pulse transmission focus point F position and the push pulse transmission transducer array Px to initial conditions, and outputs to the transmission beamformer 106 as a transmission control signal along with pulse width of a push pulse.

In step S230, the transmission beamformer 106 causes transducers included in a detection pulse transmission transducer array Tx to transmit a detection pulse pw0 to a partial region of the region of interest roi in the subject, and the reception beamformer 108 generates reference acoustic line signal frame data ds0 as a reference for tissue displacement with respect to the partial region of the region of interest roi. The reference acoustic line signal frame data ds0 is outputted to and stored by the data storage 111.

In step S140, the transmission beamformer 106 causes transformers included in the push pulse transmission transducer array Px to transmit a push pulse pp. At this time, the transmission beamformer 106 generates an initial transmission profile based on a transmission control signal set in step S120, and when second and subsequent push pulses are transmitted, generates a transmission profile based on a transmission control signal changed in step S170.

In step S250, detection pulse pwi is repeatedly transmitted and received with respect to the partial region of the region of interest roi, and an acquired acoustic line signal frame data dsi sequence is stored. An acoustic line signal frame data dsi sequence generation method is the same as that of Embodiment 1, as illustrated in FIG. 14 and FIG. 15.

In step S251, the displacement detector 109 detects displacement of an observation point Pij in a partial region of the region of interest roi for each transmission event. Details of displacement frame data pti sequence generation are the same as for Embodiment 1.

In step S252, the propagation analyzer 1101 detects a wavefront from a displacement frame data pti sequence of an observation point Pij in a partial region of the region of interest roi for each transmission event, generates an elastic modulus frame data eli sequence with respect to a partial region of the region of interest roi based on wavefront detection, and outputs to and stores in the data storage 111 (step S153). Details of elastic modulus frame data eli sequence generation are the same as for Embodiment 1, as illustrated in FIG. 12.

It is determined whether or not the processing of steps S251 to S253 is complete for all specified transmission events (step S254). If not complete, processing returns to step S251 and processing is performed for the next detection pulse transmission event. If complete, processing proceeds to step S255.

Next, the combiner 1102 combines elastic modulus frame data eli of shear waves generated with respect to partial regions of the region of interest roi and corresponding to a plurality of transmission events included in a SWS subsequence with observation points Pij as reference, calculates SWS subsequence combined elastic modulus frame data emk corresponding to the SWS subsequence (step S255), stores same in the data storage 111 (step S256), and processing proceeds to step S260.

In step S260, it is determined whether or not the processing of steps S230 to S253 is complete for all designated push pulses (step S260). If not complete, processing proceeds to step S170.

In step S170, the push pulse generator 104 changes the push pulse transmission focus point F position and the push pulse transmission transducer array Px, and outputs to the transmission beamformer 106 as a transmission control signal along with pulse width of a push pulse. Processing then proceeds to step S271. In step S271, the detection pulse generator 105 changes the detection pulse transmission focus point F position and the detection pulse transmission transducer array Tx. According to the present example, each SWS subsequence a detection pulse irradiates a partial region of the region of interest roi, and a plurality (n) of detection pulses are generated in an entire SWS sequence in order that detection pulses irradiate the entirety of the region of interest roi. More specifically, the detection pulse transmission transducer array Tx is a portion of the transducers 101a, and is gradually moved in the array direction for each SWS subsequence.

In step S271, the detection pulse generator 105 changes the detection pulse transmission focus point F position and the detection pulse transmission transducer array Tx. At this time, transmission focus point F array direction position corresponding to push pulse pp preferably coincides with a center of the detection pulse transmission transducer array Tx of a detection pulse pwi following the push pulse pp. Detection pulse transmission and reception and calculation of elastic modulus based thereon can be performed for only an area around a push pulse convergence site of the region of interest roi, processing load for one transmission event until elastic modulus calculation can be reduced, and signal acquisition time resolution can be improved.

Among detection pulse transmission focus point F positions irradiated for each SWS subsequence, transmission focus point F array direction transmission focus point position fx is gradually moved to positions that internally divide the array direction of the region of interest roi for each SWS subsequence. As a result, detection pulse pwi transmission position is gradually moved in the array direction for each SWS subsequence, in order that an entirety of the region of interest roi is irradiated by detection pulses in an entire SWS sequence. Information indicating transmission focus point F position and detection pulse transmission transducer array Tx is outputted to the transmission beamformer 106 as a transmission control signal together with pulse width of a detection pulse, and processing proceeds to step S230.

In step S260, if it is determined that processing is complete for all designated push pulses, processing proceeds to step S280.

In step S280, the subsequence combiner 1103 combines SWS subsequence combined elastic modulus frame data emk with respect to a partial region of the region of interest roi of SWS subsequences included in the SWS sequence with observation points Pij as reference, calculates SWS sequence combined elastic modulus frame data elm with respect to a partial region of the region of interest roi corresponding to the SWS sequence (step S280), and stores same in the data storage 111 (step S190). Simultaneously or alternatively, SWS sequence combined shear wave propagation speed frame data corresponding to the SWS sequence may be calculated.

This completes SWS sequence processing illustrated in FIG. 22. According to the ultrasound elastic modulus measurement processing of the ultrasonic diagnostic device 100A, SWS sequence combined elastic modulus frame data elm can be calculated.

<Effects>

As described above, according to the ultrasonic diagnostic device 100A pertaining to Embodiment 2, detection pulse pwi is transmitted and received only in the vicinity of a push pulse pp convergence site in a region of interest roi, and elastic modulus emk is calculated for a partial region of the region of interest roi for each subsequence. Thus, detection pulse pwi transmission and reception and calculation of elastic modulus based thereon can be performed for only an area around a push pulse pp convergence site of the region of interest roi, processing load for one transmission event until elastic modulus calculation can be reduced, and signal acquisition time resolution can be improved.

Further, as the push pulse pp convergence site gradually moves, detection pulse pwi transmission position is gradually moved in the array direction for each SWS subsequence, in order that detection pulse pwi is transmitted and received across the entirety of a region of interest roi, and therefore SWS sequence combined elastic modulus can be calculated with respect to the entirety of the region of interest roi corresponding to a SWS sequence.

### Embodiment 3

According to the ultrasonic diagnostic device 100 pertaining to Embodiment 1, as illustrated in FIG. 4, the detection pulse generator 105, among transmission focus point F positions, sets a depth direction transmission focus point position to a depth fz1 such that an ultrasonic beam converges at transmission focus point F at a position deeper than the region of interest roi, outside the region of interest roi, and the ultrasonic beam passes through the entirety of the region of interest roi.

However, detection pulse configuration is not limited to the above configuration, and transmission focus point F position and detection pulse transmission transducers array Tx configuration may be changed as appropriate, as long as the ultrasonic beam converges to a transmission focus point F positioned outside a region of interest roi in a subject, and the ultrasonic beam passes through the region of interest roi.

An ultrasonic diagnostic device 100B pertaining to Embodiment 3 differs from Embodiment 1 in that depth direction transmission focus point position is selected adaptively according to a measurement condition, selecting from a depth fz1 such that an ultrasonic beam converges to a transmission focus point F deeper than a region of interest roi and a depth fz3 such that the ultrasonic beam converges to a transmission focus point F shallower than the region of interest roi.

The following describes the ultrasonic diagnostic device 100B.

<Configuration>

According to the ultrasonic diagnostic device 100B, configuration of a detection pulse generated by the detection pulse generator 105 is different from the configuration of Embodiment 1, and therefore the following describes detection pulse configuration pertaining to the ultrasonic diagnostic device 100B. Configuration other than that of the detection pulse is the same as for the ultrasonic diagnostic device 100 and is not described here.

As described above, the ultrasonic diagnostic device 100B is configured to adaptively select depth direction transmission focus point position according to measurement conditions such as region of interest roi position and detection pulse transmission aperture length, selecting from depth fz1 such that an ultrasonic beam converges at a transmission focus point F position deeper than the region of interest roi and passes through the entirety of the region of interest roi and depth fz3 such that an ultrasonic beam converges at a transmission focus point F position shallower than the region of interest roi and passes through the entirety of the region of interest roi.

Of these, configuration of the detection pulse generator 105 for setting transmission focus point F as depth direction transmission focus point position fz1 is the same as described with reference to FIG. 4. As described above, among transmission focus point F positions, array direction transmission focus point position fx is calculated according to (Equation 2) and depth direction transmission focus point position fz1 is calculated according to (Equation 3).

According to the ultrasonic diagnostic device 100B, when fz1 calculated as (Equation 3) is equal to or greater than a defined threshold value, the detection pulse generator 105 sets detection pulse transmission focus point F as depth direction transmission focus point position fz3.

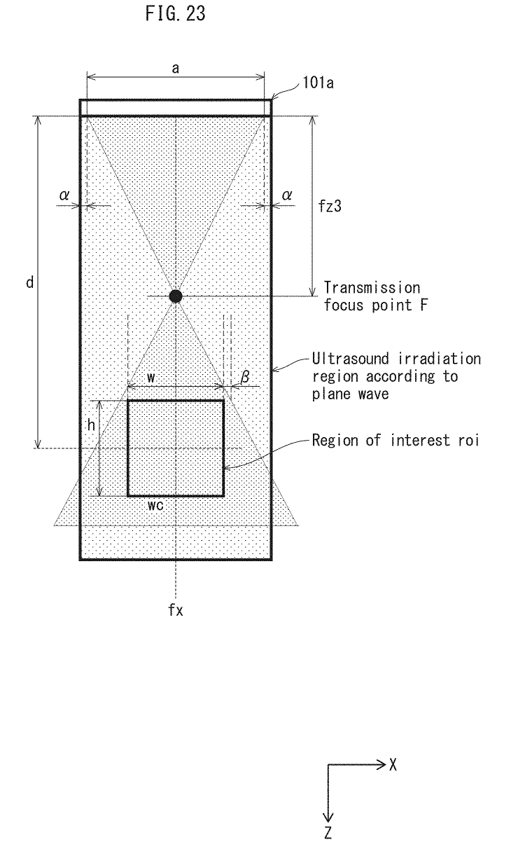

FIG. 23 is a schematic diagram illustrating a schematic configuration of a detection pulse of an ultrasonic beam converging at transmission focus point F at a position shallower than a region of interest roi, generated by the detection pulse generator 105 of the ultrasonic diagnostic device 100B. As illustrated in FIG. 22, according to the ultrasonic diagnostic device 100B, the detection pulse generator 105 sets a depth direction transmission focus point position of a detection pulse such that an ultrasonic beam converges at a transmission focus point F that is outside a region of interest roi and a position shallower than the region of interest roi and the depth direction transmission focus point position is at a depth fz3 such that the ultrasonic beam passes through the entirety of the region of interest roi. Further, in all SWS subsequences (1 to n) that constitute a SWS sequence, the detection pulse transmission transducer array Tx is all of a plurality of the transducers 101a, position of a transmission focus point F is unchanging, and the detection pulse transmission transducer array Tx is unchanging. More specifically, among transmission focus point F positions, array direction transmission focus point position fx is calculated according to (Equation 2) and depth direction transmission focus point position fz3 is calculated according to (Equation 4).

$$\begin{matrix}[ Math 4 ] & \\ fz 3 = \frac{a ( d - \frac{h}{2} )}{a + w + 2 β} & ( Equation 4 )\end{matrix}$$

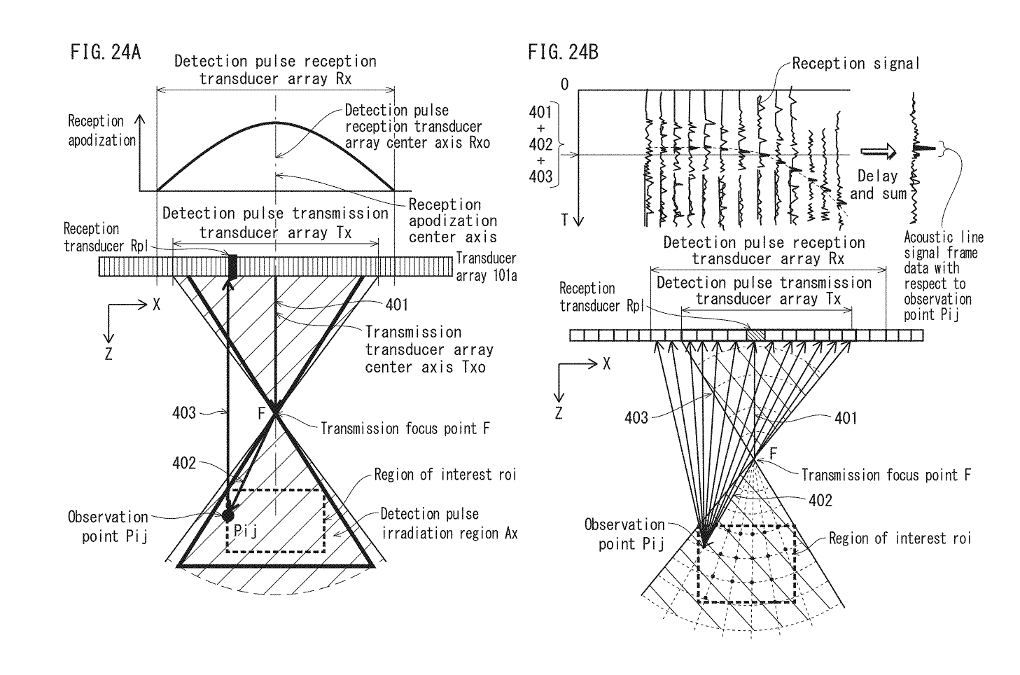

FIG. 24A and FIG. 24B are schematic diagrams for describing an outline of a reception beamforming method of the ultrasonic diagnostic device 100B and an acoustic line signal generation operation for observation point Pij in region of interest roi. As illustrated in FIG. 24A, according to the reception beamformer 108 of the ultrasonic diagnostic device 100B, a detection pulse radiated from the detection pulse transmission transducer array Tx is such that after a wavefront that travels the path 401 converges at transmission focus point F, a transmission path is assumed wherein the wavefront travels the path 402 to arrive at observation point Pij in the region of interest roi at a position deeper than the transmission focus point F, and a reception path is assumed wherein a wave reflected at the observation Pij travels the path 403 to return to a reception transducer Rpl of the probe 101. Accordingly, a value obtained by summing a travel time of a transmission wave along the path 401 and a travel time along the path 402 is the transmission time. As a specific calculation method, for example, a total path length obtained by summing length of the path 401 and length of the path 402 can be divided by propagation speed of ultrasound in a subject in order to obtain the transmission time. Then, total propagation time to a reception transducer Rpl in the detection pulse reception transducer array Rx is calculated from the transmission path and reception path. As illustrated in FIG. 24B, delay to apply to a reception signal sequence corresponding to reception transducer Rpl is calculated to perform delay-and-sum processing, and an acoustic line signal corresponding to an observation point Pij is generated. By performing this processing for every observation point Pij in a region of interest roi, acoustic line signal frame data dsi is generated for all observation points Pij in the region of interest roi.

<Operations>

The following describes operations of a SWS sequence of the ultrasonic diagnostic device 100B. According to the ultrasonic diagnostic device 100B, among ultrasound elastic modulus calculation operations of the ultrasonic diagnostic device 100 pertaining to Embodiment 1 and illustrated in FIG. 9, a detection pulse generation operation performed by the detection pulse generator 105 (step S110) is different from that of the ultrasonic diagnostic device 100, and therefore the detection pulse generation operation pertaining to the ultrasonic diagnostic device 100B is described below. Operations other than that of detection pulse generation are the same as for the ultrasonic diagnostic device 100 and not described here.

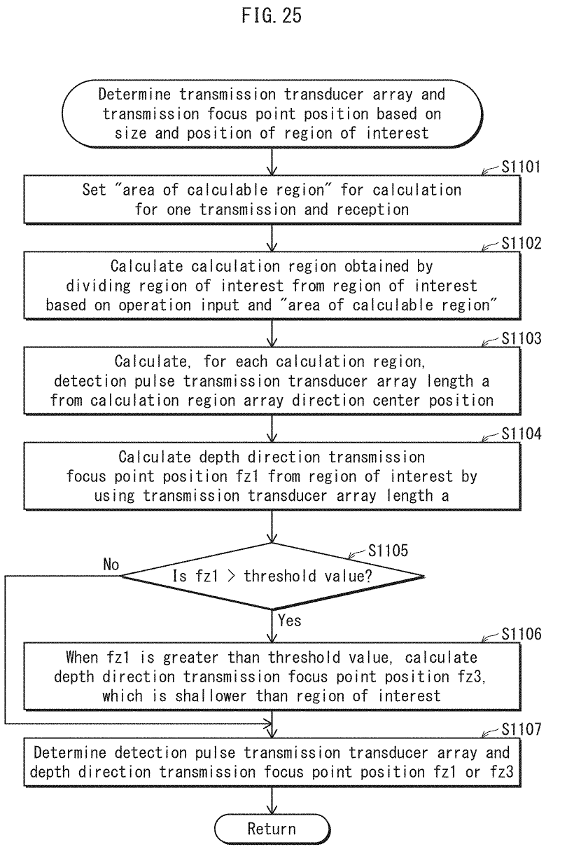

FIG. 25 is a flowchart illustrating a detection pulse generation operation of the detection pulse generator 105 in the ultrasonic diagnostic device 100B.

In steps S1101 to S1107, the detection pulse generator 105 inputs information indicating a region of interest roi from the controller 112, and adaptively selects detection pulse transmission focus point F position and detection pulse transmission transducer array Tx, based on the following conditions: size and position of region of interest roi, and detection pulse transmission aperture length.

First, the detection pulse generator 105 sets an area of a calculable region for which calculation can be performed for transmission and reception of one detection pulse. Area of a calculable region is a maximum area of a region of interest roi that can be calculated by transmission and reception of one detection pulse, and is determined according to constraint conditions of various operation modes such as emphasis on frame rate, emphasis on elastic modulus accuracy, and the like, and processing power of the controller 112 and the like.

Next, the detection pulse generator 105 inputs information indicating a region of interest roi from the controller 112, and calculates a calculation region by dividing the region of interest roi by a value obtained by dividing the area of the region of interest roi by the area of the calculable region (step S1102). The region of interest roi is inputted to the operation input unit 102 from a user as information indicating an analysis range in a subject in a previous step.

Next, the detection pulse generator 105 calculates a detection pulse transmission transducer array length a from a calculation region array direction center position for each calculation region.

Next, the detection pulse generator 105, based on the detection pulse transmission transducer array length a and the calculation region, calculates, among transmission focus point F positions, an array direction transmission focus point position fx of a detection pulse according to (Equation 2) and a depth direction transmission focus point position fz1 such that an ultrasonic beam converges to a position deeper than the region of interest roi according to (Equation 3) (step S1104).

Next, the detection pulse generator 105 determines whether or not fz1 calculated as (Equation 3) exceeds a defined threshold value (step S1104). If fz1 does not exceed the threshold value, results calculated in step S1104 are determined as depth direction transmission focus point position fz1 and detection pulse transmission transducer array Tx (step S1107).

In step S1104, if fz1 does exceed the threshold value, the detection pulse generator 105, based on the detection pulse transmission transducer array length a and the calculation region, calculates, among transmission focus point F positions, an array direction transmission focus point position fx of a detection pulse according to (Equation 2) and a depth direction transmission focus point position fz3 such that an ultrasonic beam converges to a position shallower than the region of interest roi according to (Equation 4) (step S1106). Then, calculated results are determined as depth direction transmission focus point position fz3 and detection pulse transmission transducer array Tx (step S1107).

Information indicating transmission focus point F position and detection pulse transmission transducer array Tx is outputted to the transmission beamformer 106 as a transmission control signal together with pulse width of a detection pulse (step S110).

This completes the detection pulse generation operation illustrated in FIG. 25. Thereafter, by performing processing after SWS sequence step S120, the ultrasonic diagnostic device 100B can calculate SWS sequence combined elastic modulus frame data elm according to the ultrasound elastic modulus measurement processing.

<Effects>

As described above, the ultrasonic diagnostic device 100B pertaining to Embodiment 3 is configured such that depth direction transmission focus point position is adaptively selected from a depth fz1 such that an ultrasonic beam converges to a transmission focus point F deeper than a region of interest roi and a depth fz3 such that the ultrasonic beam converges to a transmission focus point F shallower than the region of interest roi.

This configuration is effective in a situation in which depth direction transmission focus point position fz1 is too large according to the configuration of the ultrasonic diagnostic device 100, wherein detection pulse depth direction transmission focus point position fz1 is deeper than a region of interest roi and therefore only a small increase in ultrasonic beam energy density of a detection pulse in the region of interest roi is obtained and S/N improvement is small. In such a case, according to the ultrasonic diagnostic device 100B, it is possible to select detection pulse depth direction transmission focus point position fz3, which is a shallower position than a region of interest roi, and therefore detection pulse ultrasonic beam energy density in the region of interest roi can be increased, and obtained signal S/N can be improved. Thus, according to the ultrasonic diagnostic device 100B, the detection pulse generator 105 can cause transmission of a detection pulse that converges outside a region of interest and passes through the region of interest, and thereby more reliably increase ultrasonic beam energy density.

<Other Modifications>

The present invention is described based on the embodiments above, but the present invention is not limited to these embodiments, and the following modifications are also included in the scope of the present invention.

For example, the present invention may be a computer system including a microprocessor and a memory, the memory storing a computer program and the microprocessor operating according to the computer program. For example, the present invention may be a computer system that operates (or instructs operation of connected elements) according to a computer program of a diagnostic method of an ultrasonic diagnostic device of the present invention.

Further, cases in which all or part of the ultrasonic diagnostic device, or all or part of a beamforming section are constituted by a computer system including a microprocessor, a storage medium such as ROM, RAM, etc., a hard disk unit, and the like, are included in the present invention. A computer program for achieving the same operations as the devices described above may be stored in RAM or a hard disk unit. The microprocessor operating according to the computer program, thereby achieving the functions of each device.

Further, all or part of the elements of each device may be configured as one system large scale integration (LSI). A system LSI is an ultra-multifunctional LSI manufactured by integrating a plurality of elements on one chip, and more specifically is a computer system including a microprocessor, ROM, RAM, and the like. The plurality of elements can be integrated on one chip, or a portion may be integrated on one chip. Here, LSI may refer to an integrated circuit, a system LSI, a super LSI, or an ultra LSI, depending on the level of integration. A computer program for achieving the same operation as the devices described above may be stored in the RAM. The microprocessor operates according to the computer program, the system LSI thereby achieving the functions. For example, a case of the beamforming method of the present invention stored as a program of an LSI, the LSI inserted into a computer, and a defined program (beamforming method) being executed is also included in the present invention.

Note that methods of circuit integration are not limited to LSI, and implementation may be achieved by a dedicated circuit or general-purpose processor. After LSI manufacture, a field programmable gate array (FPGA) or a reconfigurable processor, in which circuit cell connections and settings in the LSI can be reconfigured, may be used.

Further, if a circuit integration technology is introduced that replaces LSI due to advances in semiconductor technology or another derivative technology, such technology may of course be used to integrate the function blocks.

Further, all or part of the functions of an ultrasonic diagnostic device pertaining to at least one embodiment may be implemented by execution of a program by a processor such as a CPU. All or part of the functions of an ultrasonic diagnostic device pertaining to at least one embodiment may be implemented by a non-transitory computer-readable storage medium on which a program is stored that causes execution of a diagnostic method or beamforming method of an ultrasonic diagnostic device described above. A program and signals may be recorded and transferred on a storage medium so that the program may be executed by another independent computer system, or the program may of course be distributed via a transmission medium such as the Internet.

According to an ultrasonic diagnostic device pertaining to at least one embodiment, the ultrasonic diagnostic device includes a data storage as a storage device. However, a storage device is not limited to this and a semiconductor memory, hard disk drive, optical disk drive, magnetic storage device, or the like may be externally connectable to the ultrasonic diagnostic device.

Further, the division of function blocks in the block diagrams is merely an example, and a plurality of function blocks may be implemented as one function block, one function block may be divided into a plurality, and a portion of a function may be transferred to another function block. Further, a single hardware or software element may process the functions of a plurality of function blocks having similar functions in parallel or by time division.

Further, the order in which steps described above are executed is for illustrative purposes, and the steps may be in an order other than described above. Further, a portion of the steps described above may be executed simultaneously (in parallel) with another step.

Further, the ultrasonic diagnostic device is described as having an externally connected probe and display, but may be configured with an integral probe and/or display.

Further, according to at least one embodiment above, the probe is configured to have a plurality of piezoelectric transducers arranged in a one-dimensional direction. However, probe configuration is not limited to this example, and as further examples, a two-dimensional transducer array in which piezoelectric transducers are arranged in a two-dimensional direction or a dynamic probe in which transducers arranged in a one-dimensional direction are mechanically swung to acquire a three-dimensional tomographic image may be used, and such probes may be used situationally depending on a measurement. For example, when a two-dimensionally arranged probe is used it is possible to control irradiation position and direction of an ultrasonic beam to be transmitted by changes to voltage application timing and value to individual piezoelectric transducers.

Further, a portion of functions of the transmitter and the detection wave receiver may be included in the probe. For example, a transmission electrical signal may be generated and converted to ultrasound in the probe, based on a control signal for generating a transmission electrical signal outputted from the transmitter. It is possible to use a configuration that converts received reflected ultrasound into a reception signal and generates an acoustic line signal based on the reception signal in the probe.

According to at least one embodiment, a configuration is described in which the probe 101 for transmitting and receiving a detection pulse pwi transmits a push pulse pp to generate a shear wave in a subject by acoustic radiation force. However, the means for generating a shear wave in a subject is not limited to transmission of a push pulse pp from the transducers 101a of the probe 101. For example, apart from the transducers 101a for performing detection pulse pwi transmission and reception, the probe 101 may be provided with ultrasound transducers for acoustic radiation force generation. Alternatively, the probe 101 may be provided with a mechanical external force generating means for radiating pressure generation, such as a vibration mechanism according to a piezoelectric element or the like. Alternatively, in addition to the probe 101 for performing transmission and reception of a detection pulse pwi, a separate probe may be provided with ultrasound transducers for acoustic radiation force generation or a mechanical external force generating means for radiating pressure generation and be connectable to the ultrasonic diagnostic device or the probe 101.

According to the ultrasonic diagnostic device 100 pertaining to at least one embodiment, configuration of the transmission beamformer 106 and the reception beamformer 108 can be changed as appropriate to configurations other than described as an embodiment.

For example, according to Embodiment 2, the transmission beamformer 106 is configured to set a transmission transducer array consisting of a transmission transducer array corresponding to a portion of the transducers 101a of the probe 101, to repeat ultrasound transmission while gradually moving the transmission transducer array in the array direction for each ultrasound transmission, and to perform ultrasound transmission from all the transducers 101a of the probe 101.

However, the transmission beamformer 106 may be configured to perform ultrasound transmission from all the transducers 101a of the probe 101. Thus, it is possible to receive reflected ultrasound from an entire ultrasound irradiated region in one ultrasound transmission without repeating ultrasound transmission.

According to Embodiments 1 and 3, the transmission beamformer 106 performs ultrasound transmission from all of the transducers 101a of the probe 101. However, the transmission beamformer 106 may set a transmission transducer array consisting of a transmission transducer array corresponding to a portion of the transducers 101a of the probe 101, repeat ultrasound transmission while gradually moving the transmission transducer array in the array direction for each ultrasound transmission, and perform ultrasound transmission from all of the transducers 101a of the probe 101. Detection pulse transmission and reception and calculation of elastic modulus based thereon can be performed for only an area around a push pulse convergence site of the region of interest roi, processing load for one transmission event until elastic modulus calculation can be reduced, and signal acquisition time resolution can be improved.

Further, according to at least one embodiment, a region in which observation points exist is a linear region having a width of a single transducer, perpendicular to the transducer array and passing through a center of a reception transducer array.

However, the present invention is not limited to this, and the region may be set to any region included in an ultrasound irradiation region. For example, a rectangular region having a width of a plurality of transducers and a center line of a straight line perpendicular to a reception transducer array that passes through a center of the reception transducer array may be used.

Further, at least a portion of functions of each ultrasonic diagnostic device pertaining to an embodiment, and each modification thereof, may be combined. Further, the numbers used above are all illustrative, for the purpose of explaining the present invention in detail, and the present invention is not limited to the example numbers used above. Further, the present invention includes various modifications that are within the scope of conceivable ideas by a person skilled in the art.

<<Summary>>

An ultrasonic diagnostic device pertaining to at least one embodiment is an ultrasonic diagnostic device that causes a probe to transmit a push pulse converging on a specific site in a subject and detects propagation speed of a shear wave generated by acoustic radiation force of the push pulse, the probe being connectable to the ultrasonic diagnostic device and including transducers arranged along an array direction, the ultrasonic diagnostic device comprising: an operation input unit that receives operation input; a region of interest setter that sets a region of interest representing a range of analysis in the subject, based on the operation input; a push pulse generator that sets the specific site in the subject and causes the transducers to transmit the push pulse; a detection pulse generator that, following the push pulse, causes a portion of or all of the transducers to transmit detection pulses that each converge outside the region of interest and pass through the region of interest; a reception beamformer that generates an acoustic line signal frame data sequence by generating acoustic line signals with respect to observation points in the region of interest, based on reflected detection waves corresponding to the detection pulses that are reflected from tissue of the subject and received in a time sequence by the transducers; and an elastic modulus calculator that detects tissue displacement in the region of interest from the acoustic line signal frame data sequence, generates a wavefront frame data sequence representing shear wave wavefront position at time points on a time axis each corresponding to one of the detection pulses, and calculates shear wave propagation speed and/or elastic modulus frame data for the region of interest based on wavefront position changes and time intervals between the wavefront frame data frames.

In ultrasound elastic modulus measurement according to a SWS sequence, in order to improve signal S/N, a region of interest including a site of elastic modulus measurement is usually set near or around a push pulse transmission focus point. In contrast, according to the configuration above, by adopting a configuration in which a detection pulse pwi that converges outside a region of interest roi in a subject and passes through the region of interest roi is transmitted and reflected detection waves are received, it is possible for the detection pulse to irradiate a region of interest including a site of elastic modulus measurement without excess or shortfall, and to calculate elastic modulus based on reception thereof. Thus, ultrasonic beam energy density of a detection pulse in a region of interest can be increased, and in ultrasound elastic modulus measurement, signal acquisition time resolution and signal S/N for elasticity image generation can be improved over conventional technology using a plane wave as a detection pulse.

According to another embodiment, the detection pulse generator causes the detection pulses to be transmitted so as to converge to a position deeper than the region of interest in an ultrasound transmission direction in the subject.

According to this configuration, detection pulse ultrasonic beam energy density can be increased in a region of interest roi and signal S/N for elasticity image generation can be improved when the region of interest roi is positioned relatively deep in the depth direction of the subject.

According to another embodiment, the detection pulse generator causes the detection pulses to be transmitted so as to converge to a position shallower than the region of interest in an ultrasound transmission direction in the subject.

According to this configuration, detection pulse ultrasonic beam energy density can be increased in a region of interest roi and signal S/N for elasticity image generation can be improved when the region of interest roi is positioned relatively shallowly in the depth direction of the subject.

According to another embodiment, the detection pulse generator determines transmission transducers to transmit the detection pulses and depth at which the detection pulses converge in the subject such that a detection pulse ultrasonic beam passes through an entirety of the region of interest.

According to this configuration, an acoustic line signal can be generated for observation points in the entire region of interest by transmission and reception of one detection pulse, and therefore in ultrasound elastic modulus measurement signal acquisition time resolution can be improved.

According to another embodiment, the region of interest is within a range sandwiched between two straight lines that connect both ends in the array direction of the transmission transducers to a beam center at the depth at which the detection pulses converge in the subject.

According to this configuration, the detection pulse can be transmitted so that an ultrasonic beam reliably passes through the entire region of interest.

According to another embodiment, the detection pulse generator determines transmission transducers to transmit the detection pulses and depth at which the detections pulses converge in the subject such that a detection pulse ultrasonic beam passes through a partial region of the region of interest.

According to this configuration, detection pulse transmission and reception and calculation of elastic modulus based thereon can be performed for only an area around a push pulse convergence site of the region of interest, processing load for one transmission event until elastic modulus calculation can be reduced, and signal acquisition time resolution can be improved.

According to another embodiment, the detection pulse generator determines transmission transducers to transmit the detection pulses and depth at which the detections pulses converge in the subject based on depth of the region of interest in the subject and size of the region of interest in the array direction.

According to this configuration, the detection pulse generator can determine transmission transducers to transmit detection pulses and depth at which the detections pulses converge in the subject as appropriate.

According to another embodiment, the detection pulse generator causes the detection pulses to converge at a position deeper than the region of interest in the subject in an ultrasound transmission direction when a parameter based on depth of the region of interest in the subject, size of the region of interest in the array direction, and number of transmission transducers transmitting the detection pulses is less than or equal to a threshold value, and causes the detection pulses to converge at a position shallower than the region of interest in the subject in the ultrasound transmission direction when the parameter is greater than the threshold value.

According to this configuration, when the detection pulse depth direction transmission focus point position is deeper than the region of interest and exceeds the threshold value, a situation can be prevented in which only a small increase in detection pulse ultrasonic beam energy density in the region of interest and a small improvement in signal S/N is obtained. In other words, it is possible to select a detection pulse depth direction transmission focus point position that is shallower than the region of interest, and therefore detection pulse ultrasonic beam energy density in the region of interest roi can be increased, and obtained signal S/N can be improved.

According to another embodiment, the threshold value is 2, and the parameter is calculated as fz1 by

$$\begin{matrix}[ Math 3 ] & \\ fz 1 = \frac{a ( d + \frac{h}{2} )}{a - w - 2 β} & ( Equation 3 )\end{matrix}$$

where a is length in the array direction of an array of the transmission transducers for transmitting the detection pulses, d is depth from a surface of the subject to a center of the region of interest, h is length of the region of interest in a depth direction of the subject, w is length of the region of interest in the array direction, and β is distance in the array direction between the region of interest and either of two straight lines that connect both ends in the array direction of the array of the transmission transducers to a beam center at the depth at which the detection pulses converge in the subject.

According to this configuration, depth direction transmission focus point position can be adaptively selected according to measurement conditions from a depth fz1 at which an ultrasonic beam converges at a transmission focus point F deeper than the region of interest and a depth fz3 at which an ultrasonic beam converges at a transmission focus point F shallower than the region of interest. Thus, the detection pulse generator can cause transmission of a detection pulse that converges outside a region of interest and passes through the region of interest, and thereby more reliably increase ultrasonic beam energy density.

According to another embodiment, the push pulse generator sets the specific site multiple times, each time at a different position in the region of interest of the subject, and causes transmission of the push pulse multiple times, converging on the different positions, the detection pulse generator causes transmission of the detection pulses following each transmission of the push pulse, the reception beamformer generates a plurality of acoustic line signal frame data sequences, each sequence corresponding to transmission of the detection pulses following a transmission of the push pulse, and the elastic modulus calculator calculates, from the plurality of acoustic line signal frame data sequences, shear wave propagation speed and/or elastic modulus frame data corresponding to each transmission of the push pulse with respect to the observation points in the region of interest, and calculates combined shear wave propagation speed and/or combined elastic modulus frame data with respect to the observation points in the region of interest by using each of the observation points as an index and summing corresponding calculated propagation speed and/or elastic modulus frame data. According to another embodiment, a center in the array direction of an array of the transducers transmitting the detection pulses coincides with a center in the array direction of the region of interest.

According to this configuration, propagation speed and/or elastic modulus frame data calculated corresponding to a plurality of push pulses in a SWS sequence can be summed using positions of observation points as an index, and therefore signal S/N for elasticity image generation can be improved.

According to another embodiment, the push pulse generator sets the specific site multiple times, each time at a different position in the region of interest of the subject, and causes transmission of the push pulse multiple times, converging on the different positions, the detection pulse generator causes only a portion of the transducers to transmit the detection pulses following each transmission of the push pulse, and each of the detection pulses passes through only a partial region of the region of interest, the reception beamformer generates acoustic line signals with respect to each observation point in each partial region of the region of interest in order to generate a plurality of acoustic line signal frame data sequences, each sequence corresponding to transmission of the detection pulses following a transmission of the push pulse, and the elastic modulus calculator calculates, from the plurality of acoustic line signal frame data sequences, shear wave propagation speed and/or elastic modulus frame data corresponding to each transmission of the push pulse with respect to each plurality of observation points in each partial region of the region of interest, and calculates combined shear wave propagation speed and/or combined elastic modulus frame data with respect to the observation points in the region of interest by using each of the observation points as an index and summing corresponding calculated propagation speed and/or elastic modulus frame data. According to another embodiment, a center in the array direction of the specific site corresponding to the push pulse coincides with a center in the array direction of an array of the transducers transmitting the detection pulses following the push pulse.

According to this configuration, as a push pulse convergence site is gradually moved, detection pulse transmission position is gradually moved for each SWS subsequence to transmit and receive detection pulses to the entirety of the region of interest, and therefore SWS sequence combined elastic modulus can be calculated for the entirety of the region of interest corresponding to a SWS sequence. Further, detection pulse transmission and reception and calculation of elastic modulus based thereon can be performed for only an area around a push pulse convergence site of the region of interest, and therefore processing load for one transmission event until elastic modulus calculation can be reduced and signal acquisition time resolution can be improved.

According to another embodiment, the reception beamformer includes: an input unit that, based on reflected detection waves reflected from the tissue of the subject and received in a time sequence by the transducers, generates a reception signal sequence for each of the transducers; and a delay-and-sum unit that generates the acoustic line signals for each of the observation points in the region of interest by performing delay-and-sum processing on the reception signal sequences.

According to this configuration, it is possible to generate an acoustic line signal even based on reflected detection waves from the entirety of the region of interest from one detection pulse transmission event, which can improve efficiency of detection pulse transmission. Thus, signal S/N for elasticity image generation can be improved.

According to another embodiment, the delay-and-sum unit, from a sum of transmission time of the detection pulses from transmission to arrival at an observation point in the region of interest and reception time of reflected waves from the observation point to each of the transducers, calculates total propagation time for transmitted ultrasonic reflected at the observation point to arrive at each of the transducers, and calculates a delay for each of the transducers based on the total propagation time, identifies a reception signal value corresponding to a delay for each of the transducers from the reception signal sequence corresponding to the transducer, and sums the reception signal values to generate an acoustic line signal for the observation point.

According to this configuration, delay-and-sum focusing on each observation point in the region of interest is performed by delay processing based on total propagation time, making it possible to generate an acoustic line signal for each observation point.

According to another embodiment, a distance between a center in the array direction of an array of transducers transmitting the detection pulses and a beam center at a depth where the detection pulses converge in the subject is a first distance and a distance between the beam center and an observation point in the region of interest is a second distance, the delay-and-sum unit, when the region of interest is deeper in the subject depth direction than the beam center, calculates the transmission time by dividing a sum of the first distance and the second distance by a speed of sound, and when the region of interest is shallower in the subject depth direction than the beam center, calculates the transmission time by dividing a difference obtained by subtracting the second distance from the first distance by the speed of sound.

According to this configuration, depth direction transmission focus point position can be adaptively selected according to measurement conditions from a depth fz1 at which an ultrasonic beam converges at a transmission focus point F deeper than the region of interest and a depth fz3 at which an ultrasonic beam converges at a transmission focus point F shallower than the region of interest. In each case, delay processing is performed based on total propagation path, and therefore focused delay-and-sum is performed with respect to each of the observation points in the region of interest, and acoustic line signals can be generated with respect to each of the observation points.

According to another embodiment, the elastic modulus calculator calculates shear wave propagation speed and/or elastic modulus frame data sequences corresponding to the observation points in the region of interest a plurality of times from the acoustic line signal frame data sequences, and by summing frame data sequences with positions of the observation points as an index, combines shear wave propagation speed and/or elastic modulus frame data corresponding to each transmission of the push pulse.

According to this configuration, propagation speed and/or elastic modulus frame data calculated corresponding to detection pulses in SWS subsequences can be summed with observation point position as an index by a combining aperture method, and therefore even for observation points at depths other than a transmission focus point F of a plurality of transmission events, results of virtual transmission focus can be obtained, and spatial resolution and signal S/N can be further improved. Thus, signal S/N for elasticity image generation can be improved.

According to another embodiment, the ultrasonic diagnostic device further comprises a display that displays an image, wherein the elastic modulus calculator generates an elasticity image by mapping the elastic modulus frame data, converts the elasticity image to a display image, and causes the display to display the display image.

According to this configuration, it is possible to display intensity distribution of elastic modulus frame data in the region of interest detected by ultrasound elastic modulus measurement in an easy-to-view manner.

An ultrasonic signal processing method pertaining to at least one embodiment is an ultrasonic signal processing method for causing a probe to transmit a push pulse converging on a specific site in a subject and detecting propagation speed of a shear wave generated by acoustic radiation force of the push pulse, the probe being connectable to the ultrasonic diagnostic device and including transducers arranged along an array direction, the ultrasonic signal processing method comprising: receiving operation input; setting a region of interest representing a range of analysis in the subject, based on the operation input; setting the specific site in the subject and causing the transducers to transmit the push pulse; following the push pulse, causing a portion of or all of the transducers to transmit detection pulses that each converge outside the region of interest and pass through the region of interest; generating an acoustic line signal frame data sequence by generating acoustic line signals with respect to observation points in the region of interest, based on reflected detection waves corresponding to the detection pulses that are reflected from the tissue of the subject and received in a time sequence by the transducers; and detecting tissue displacement in the region of interest from the acoustic line signal frame data sequence, generating a wavefront frame data sequence representing shear wave wavefront position at time points on a time axis each corresponding to one of the detection pulses, and calculating shear wave propagation speed and/or elastic modulus frame data for the region of interest based on wavefront position changes and time intervals between the wavefront frame data frames. The ultrasonic signal processing method may be implemented as a computer-readable non-transitory storage medium on which the ultrasonic signal processing method is stored.

According to this configuration, in ultrasound elastic modulus measurement, signal acquisition time resolution and signal S/N for elasticity image generation can be improved over conventional art using a plane wave detection pulse.

<<Supplement>>

The embodiments described above each indicate one preferred specific example of the present invention. Numerical values, shapes, materials, constituent elements, arrangement positions and connections of constituent elements, steps, order of steps, and the like indicated as embodiments are merely examples and are not intended to limit the present invention. Further, among constituent elements in the embodiments, steps not described in independent claims representing top level concepts of the present invention are described as any constituent element constituting a more preferable embodiment.

Further, in order to facilitate understanding of the invention, scale of constituent elements in each drawing referenced by description of an embodiment may differ from actual scale. Further, the present invention is not limited by the description of each embodiment, and can be appropriately changed without departing from the scope of the present invention.

Further, in the ultrasonic diagnostic device there are members such as circuit parts, lead wires, and the like on a substrate, but these may be implemented in various forms based on ordinary knowledge in technical fields concerning electric wiring and electric circuits, and are not directly relevant to description of the present invention, and therefore description thereof is omitted herein. Note that each drawing is a schematic diagram and is not necessarily a strict depiction.

### INDUSTRIAL APPLICABILITY

The ultrasonic signal processing circuitry, ultrasonic diagnostic device, ultrasonic signal processing method, and computer-readable non-transitory storage medium pertaining to the present disclosure are useful for improving performance of a conventional ultrasonic diagnostic device, in particular for improving image quality. In addition, the present disclosure can be applied not only to ultrasound, but also to applications such as sensors using a plurality of array transducers.

### REFERENCE SIGNS LIST

100, 100A, 100B ultrasonic diagnostic device
101 probe
101a ultrasound transducers
102 operation input unit
103 region of interest setter
104 push pulse generator
105 detection pulse generator
106 transmission beamformer
1061 drive signal generator
1062 delay profile generator
1063 drive signal transmitter
107 multiplexer
108 reception beamformer
1081 input unit
1082 reception signal holding unit
1083 delay-and-sum unit
10831 delay processing section
10832 summing section
109 displacement detector
110 elastic modulus calculator
1101 propagation information analyzer
1102 combiner
1103 subsequence combiner
111 data storage
112 controller
113 display controller
114 display
150 ultrasonic signal processing circuitry

1. An ultrasonic diagnostic device that causes a probe to transmit a push pulse converging on a specific site in a subject and detects propagation speed of a shear wave generated by acoustic radiation force of the push pulse, the probe being connectable to the ultrasonic diagnostic device and including transducers arranged along an array direction, the ultrasonic diagnostic device comprising:
an operation input unit that receives operation input;
a region of interest setter that sets a region of interest representing a range of analysis in the subject, based on the operation input;
a push pulse generator that sets a transmission focus point corresponding to the specific site in the subject and causes the transducers to transmit the push pulse;
a detection pulse generator that, following the push pulse, causes a portion of or all of the transducers to transmit detection pulses that each converge outside the region of interest and pass through the region of interest;
a reception beamformer that generates an acoustic line signal frame data sequence by generating acoustic line signals with respect to observation points in the region of interest, based on reflected detection waves corresponding to the detection pulses that are reflected from tissue in the subject and received in a time sequence by the transducers; and
an elastic modulus calculator that detects tissue displacement in the region of interest from the acoustic line signal frame data sequence, generates a wavefront frame data sequence representing shear wave wavefront position at time points on a time axis each corresponding to one of the detection pulses, and calculates shear wave propagation speed and/or elastic modulus frame data for the region of interest based on wavefront position changes and time intervals between the wavefront frame data frames, wherein
the detection pulse generator determines whether to cause the detection pulses to converge at a position deeper than the region of interest in the subject in an ultrasound transmission direction or cause the detection pulses to converge at a position shallower than the region of interest in the subject in the ultrasound transmission direction, according to depth of the region of interest in the subject, size of the region of interest in the array direction, and number of transmission transducers transmitting the detection pulses.

2. (canceled)

3. (canceled)

4. The ultrasonic diagnostic device according to claim 1, wherein
the detection pulse generator determines transmission transducers to transmit the detection pulses and depth at which the detection pulses converge in the subject such that a detection pulse ultrasonic beam passes through an entirety of the region of interest.

5. The ultrasonic diagnostic device according to claim 4, wherein
the region of interest is within a range sandwiched between two straight lines that connect both ends in the array direction of the transmission transducers to a beam center at the depth at which the detection pulses converge in the subject.

6. The ultrasonic diagnostic device according to claim 1, wherein
the detection pulse generator determines transmission transducers to transmit the detection pulses and depth at which the detections pulses converge in the subject such that a detection pulse ultrasonic beam passes through a partial region of the region of interest.

7. (canceled)

8. The ultrasonic diagnostic device according to claim 1, wherein
the detection pulse generator causes the detection pulses to converge at a position deeper than the region of interest in the subject when a parameter based on the depth of the region of interest in the subject, the size of the region of interest in the array direction, and the number of transmission transducers transmitting the detection pulses is less than or equal to a threshold value, and causes the detection pulses to converge at a position shallower than the region of interest in the subject in the ultrasound transmission direction when the parameter is greater than the threshold value.

9. The ultrasonic diagnostic device according to claim 8, wherein
the threshold value is 2, and the parameter is calculated as fz1 by
$$\begin{matrix}[ Math 1 ] & \\ fz 1 = \frac{a ( d + \frac{h}{2} )}{a - w - 2 β} & ( Equation 1 )\end{matrix}$$
where a is length in the array direction of an array of the transmission transducers for transmitting the detection pulses, d is depth from a surface of the subject to a center of the region of interest, h is length of the region of interest in a depth direction of the subject, w is length of the region of interest in the array direction, and β is distance in the array direction between the region of interest and either of two straight lines that connect both ends in the array direction of the array of the transmission transducers to a beam center at the depth at which the detection pulses converge in the subject.

10. The ultrasonic diagnostic device according to claim 1, wherein
the push pulse generator sets the transmission focus point corresponding to the specific site multiple times, each time at a different position in the region of interest of the subject, and causes transmission of the push pulse multiple times, converging on the different positions,
the detection pulse generator causes transmission of the detection pulses following each transmission of the push pulse,
the reception beamformer generates a plurality of acoustic line signal frame data sequences, each sequence corresponding to transmission of the detection pulses following a transmission of the push pulse, and
the elastic modulus calculator calculates, from the plurality of acoustic line signal frame data sequences, shear wave propagation speed and/or elastic modulus frame data corresponding to each transmission of the push pulse with respect to the observation points in the region of interest, and calculates combined shear wave propagation speed and/or combined elastic modulus frame data with respect to the observation points in the region of interest by using each of the observation points as an index and summing corresponding calculated propagation speed and/or elastic modulus frame data.

11. The ultrasonic diagnostic device according to claim 1, wherein
a center in the array direction of an array of the transducers transmitting the detection pulses coincides with a center in the array direction of the region of interest.

12. The ultrasonic diagnostic device according to claim 1, wherein
the push pulse generator sets the transmission focus point corresponding to the specific site multiple times, each time at a different position in the region of interest of the subject, and causes transmission of the push pulse multiple times, converging on the different positions,
the detection pulse generator causes only a portion of the transducers to transmit the detection pulses following each transmission of the push pulse, and each of the detection pulses passes through only a partial region of the region of interest,
the reception beamformer generates acoustic line signals with respect to each observation point in each partial region of the region of interest in order to generate a plurality of acoustic line signal frame data sequences, each sequence corresponding to transmission of the detection pulses following a transmission of the push pulse, and
the elastic modulus calculator calculates, from the plurality of acoustic line signal frame data sequences, shear wave propagation speed and/or elastic modulus frame data corresponding to each transmission of the push pulse with respect to each plurality of observation points in each partial region of the region of interest, and calculates combined shear wave propagation speed and/or combined elastic modulus frame data with respect to the observation points in the region of interest by using each of the observation points as an index and summing corresponding calculated propagation speed and/or elastic modulus frame data.

13. The ultrasonic diagnostic device according to claim 12, wherein
a center in the array direction of the transmission focus point corresponding to the specific site corresponding to the push pulse coincides with a center in the array direction of an array of the transducers transmitting the detection pulses following the push pulse.

14. The ultrasonic diagnostic device according to claim 1, wherein
the reception beamformer includes:
an input unit that, based on reflected detection waves reflected from the tissue in the subject and received in a time sequence by the transducers, generates a reception signal sequence for each of the transducers; and
a delay-and-sum unit that generates the acoustic line signals for each of the observation points in the region of interest by performing delay-and-sum processing on the reception signal sequences.

15. The ultrasonic diagnostic device according to claim 14, wherein
the delay-and-sum unit,
from a sum of transmission time of the detection pulses from transmission to arrival at an observation point in the region of interest and reception time of reflected waves from the observation point to each of the transducers,
calculates total propagation time for transmitted ultrasonic reflected at the observation point to arrive at each of the transducers, and calculates a delay for each of the transducers based on the total propagation time,
identifies a reception signal value corresponding to a delay for each of the transducers from the reception signal sequence corresponding to the transducer, and sums the reception signal values to generate an acoustic line signal for the observation point.

16. The ultrasonic diagnostic device according to claim 15, wherein
a distance between a center in the array direction of an array of transducers transmitting the detection pulses and a beam center at a depth where the detection pulses converge in the subject is a first distance and a distance between the beam center and an observation point in the region of interest is a second distance,
the delay-and-sum unit,
when the region of interest is deeper in the subject depth direction than the beam center, calculates the transmission time by dividing a sum of the first distance and the second distance by a speed of sound, and
when the region of interest is shallower in the subject depth direction than the beam center, calculates the transmission time by dividing a difference obtained by subtracting the second distance from the first distance by the speed of sound.

17. The ultrasonic diagnostic device according to claim 10, wherein
the elastic modulus calculator calculates shear wave propagation speed and/or elastic modulus frame data sequences corresponding to the observation points in the region of interest a plurality of times from the acoustic line signal frame data sequences, and by summing frame data sequences with positions of the observation points as an index, combines shear wave propagation speed and/or elastic modulus frame data corresponding to each transmission of the push pulse.

18. The ultrasonic diagnostic device according to claim 1, further comprising:
a display that displays an image, wherein
the elastic modulus calculator generates an elasticity image by mapping the elastic modulus frame data, converts the elasticity image to a display image, and causes the display to display the display image.

19. An ultrasonic signal processing method for causing a probe to transmit a push pulse converging on a specific site in a subject and detecting propagation speed of a shear wave generated by acoustic radiation force of the push pulse, the probe being connectable to the ultrasonic diagnostic device and including transducers arranged along an array direction, the ultrasonic signal processing method comprising:
receiving operation input;
setting a region of interest representing a range of analysis in the subject, based on the operation input;
setting a transmission focus point corresponding to the specific site in the subject and causing the transducers to transmit the push pulse;
following the push pulse, causing a portion of or all of the transducers to transmit detection pulses that each converge outside the region of interest and pass through the region of interest;
generating an acoustic line signal frame data sequence by generating acoustic line signals with respect to observation points in the region of interest, based on reflected detection waves corresponding to the detection pulses that are reflected from tissue in the subject and received in a time sequence by the transducers;
detecting tissue displacement in the region of interest from the acoustic line signal frame data sequence, generating a wavefront frame data sequence representing shear wave wavefront position at time points on a time axis each corresponding to one of the detection pulses, and calculating shear wave propagation speed and/or elastic modulus frame data for the region of interest based on wavefront position changes and time intervals between the wavefront frame data frames, and
determining whether to cause the detection pulses to converge at a position deeper than the region of interest in the subject in an ultrasound transmission direction or cause the detection pulses to converge at a position shallower than the region of interest in the subject in the ultrasound transmission direction, according to depth of the region of interest in the subject, size of the region of interest in the array direction, and number of transmission transducers transmitting the detection pulses.
